(19) United States
(12) Patent Application Publication
(10) Pub. No.: US 2009/0176970 A1
(43) Pub. Date: Jul. 9, 2009

(54) Preparation of Thiosugars and Their Use

(76) Inventors: Benjamin Guy Davis, Cambridge (GB); Antony John Fairbanks, Cambridge (GB); Niels Christian Reichhardt, Cambridge (GB); Vinod Unni Angadiparambil, Cambridge (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/278,444

(22) PCT Filed: Feb. 6, 2007
(86) PCT No.: PCT/GB07/00398

§ 371 Date: Dec. 10, 2008

(30) Foreign Application Priority Data
Feb. 6, 2006 (GB) 0602352.7

(51) Int. Cl. C07H 1/00; C08B 37/00; C07K 14/00

(52) U.S. Cl. 530/395; 536/124; 536/123.1

(57) ABSTRACT

A process for the preparation of a thiosaccharide represented by Saccharide-S-H wherein Saccharide comprises at least 4 sugar units, comprises subjecting a corresponding compound of the formula (P)Saccharide-S-(P) wherein (P) represents an O- or S-protecting group(s), to Birch reduction.

### FIELD OF THE INVENTION

This invention relates to a method useful for producing a glycoprotein, including the synthesis of thiosugars.

### BACKGROUND OF THE INVENTION

WO2005/00862 describes a process for the coupling of a protein having SH groups with a thiosugar, using selenium chemistry. This may be represented as the reaction of the compounds Protein-S-Se-R and Saccharide-SH or Protein-SH and R-Se-S-Saccharide, to give Protein-S-S-Saccharide. R is defined as optionally substituted alkyl, phenyl, pyridyl or naphthyl.

In the synthesis of polysaccharides, the coupling of saccharide units requires selective protection of functional groups such as OH. Various protecting groups are known for this purpose, including acetyl and benzyl. The final step requires removal of such groups.

Boons et al., Chemistry & Biology (2003) 10, 87 and Carbohydrate Research (2004), 339, 181, discloses the thio-pentasaccharide which is compound 78, below. The given preparation is by reaction of glucosyl chloride with potassium thioacetate, followed by deprotection. This process is not easily reproducible.

### SUMMARY OF THE INVENTION

The present invention is based in part on the discovery that the practical application of the process described by Boons et al is limited to lower saccharides, and that the known pentasaccharide and higher saccharides can be prepared by a Birch reduction. According to a first aspect of the invention, therefore, a process for preparing a polysaccharide of the formula Saccharide-SH comprises the reaction of (P) Saccharide-S-(P), wherein (P) represents an O- or S-protecting group such as benzyl or acetyl, with an alkali metal in liquid ammonia.

Saccharide-SH can be converted to a glycoprotein by a procedure as described in WO2005/00862. R may be any suitable group, e.g. as defined above, but its precise formula is not critical, provided that the relevant reaction can occur. Alternatively, according to a further aspect of the invention, it may be allowed to dimerise, to give a dimer of the formula Saccharide-S-S-Saccharide, and then preparation of the glycoprotein comprises the reaction of a protein containing one or more available SH groups with the dimer.

The thio decasaccharide 47 and its dimer 48 are novel compounds, and they constitute further aspects of this invention.

### DESCRIPTION OF PREFERRED EMBODIMENTS

The blocked reactant used in this invention (illustrated by compounds 46 and 77, below) typically has all OH and SH groups blocked as an ester or ether. The protecting group may be aliphatic or aromatic, and may have up to 12 C atoms. Preferred examples are acetyl and benzyl.

The reactant may also include blocked amino groups. They are not necessarily affected by the Birch reduction.

The reactant and product have at least 4, preferably at least 5, e.g. up to 10, or 20 saccharide units.

The Birch reduction utilises an alkali metal such as Li, Na or K, preferably Na, in liquid ammonia. This reaction effectively removes the protecting groups.

As indicated above, the product of the novel reaction may be converted to a glycoprotein, using a process as described in WO2005100862. Alternatively, prior to reaction with Protein-SH, the dimer may be prepared, e.g. by simply exposing an aqueous solution of the thiosugar to air.

The glycoprotein that is produced by the invention may have one or more, e.g. 2, 3 or 4, -S-S-Saccharide groups. As will be readily understood by one of ordinary skill in the art, the number of such groups will depend on the number of available SH groups on Protein.

Some proteins have available SH groups. Those that do not may be modified to include one or more Cys residues; Protein-SH for use in the invention may be prepared by site-directed mutagenesis, e.g. as described in WO2005/00862. Examples of useful proteins that can be modified are erythropoietin and glucocerebrosidase. In the Examples, below, a mutant of SBL, S156C, is used as a model protein.

The following Examples illustrate the invention. In particular, they describe:

1) decasaccharide 47, its dimer (compound 48) and coupling, following the respective synthesis of fragments represented by trisaccharides 7, 12 and 29; and
2) pentasaccharide 78, its dimer (compound 79) and coupling, following the respective synthesis of disaccharides 60 and 70.

The following abbreviations are used:

OTCA trichloroacetimidate

Lev levulinyl

PMP para-methoxyphenyl

NPhth phthalimido

Pent 4-pentenyl

TBDMS tert-butyldimethylsilyl

Petrol refers to the fraction boiling in the range 40° C.-60° C.

### Compound 1

### Benzyl α-D-mannopyranoside

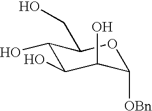

D-Mannose (60 g) was added to benzyl alcohol (400 mL.) After 24 hours, acetyl chloride (24 mL) was added and the mixture stirred at 60° C. for 2 hours. The mixture was then allowed to stand overnight at room temperature. The mixture was then heated at 116° C. under reduced pressure (approximately 10 mbar) to remove excess benzyl alcohol. Dry flash chromatography [SiO₂, ethyl acetate:methanol (100:0) to (92:8)] gave a solid that was recrystallised from isopropanol-petrol to give compound 1 (26.6 g).

### Compound 2

### Benzyl 3,4-O-(2′,3′-dimethoxybutane-2′,3′-diyl)-α-D-mannopyranoside

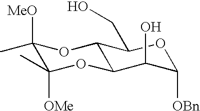

Butane-2,3-dione (1.27 g), trimethyl orthoformate (39.3 g) and camphorsulfonic acid (1.72 g) were added to a mixture of compound 1 (20 g) in dry methanol (200 mL) and the mixture refluxed under argon for 16 hours. Triethylamine (50 mL) was then added followed by water (200 mL) and dichloromethane (300 mL). The organic phase was separated and the aqueous phase was extracted with dichloromethane (2×200 mL). The combined organic fractions were dried (MgSO₄) and concentrated under reduced pressure. Chromatography [SiO₂, ethyl acetate:petrol (80:20)] gave compound 2 (20.2 g).

### Compound 3

### 1,3,4,6-Tetra-O-acetyl-2-deoxy-2-phthalimido-D-glucopyranose

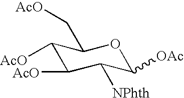

Sodium methoxide (13.5 g) was added to a stirred suspension of D-(+)-glucosamine hydrochloride (53.9 g) in methanol. After 1 hour the reaction was placed in a cold water bath and phthalic anhydride (36.9 g) added. Triethylamine (34.7 mL) was then added over 15 minutes. After 2 days the precipitate was collected by filtration and washed with methanol (2×20 ml) and evaporated under reduced pressure. Pyridine (675 mL) was added and the stirred mixture cooled to 0° C. and acetic anhydride (675 mL) added over 1 hour. After 1 day at room temperature the mixture was cooled to 0° C. and ethanol (270 mL) added over 30 minutes. After 3 hours the mixture was evaporated under reduced pressure and dissolved in dichloromethane (1 L). The mixture was washed with 1M hydrochloric acid (2×500 mL) and saturated sodium hydrogen carbonate (2×500 mL), dried (MgSO₄) and evaporated under reduced pressure. Recrystallisation from methanol gave compound 3 (41.8 g). The mother liquors were evaporated under reduced pressure and chromatographed [SiO₂, ethyl acetate:petrol (50:50) to give further compound 3 (35.1 g).

### Compound 4

### Phenylselenyl-3,4,6-tri-O-acetyl-1,2-deoxy-2-phthalimido-β-D-glucopyranose

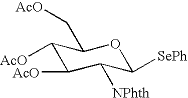

Boron trifluoride etherate (17.5 mL) was added to a stirred solution of compound 3 (52.4 g) and phenylselenol (14.6 mL) in dry dichloromethane (750 mL) under argon. After 20 hours triethylamine (30 mL) was added dropwise. The mixture was then washed with water (2×250 mL), dried (MgSO₄) and evaporated under reduced pressure. Chromatography [SiO₂, ethyl acetate:petrol (50:50) gave a product that was recrystallised from ethyl acetate-petrol to give compound 4 (44 g).

### Compound 5

### Benzyl 3,4,6-tri-O-acetyl-2-deoxy-2-phthalimido-β-D-glucopyranosyl-(1→6)-[3,4,6-tri-O-acetyl-2-deoxy-2-phthalimido-β-D-glucopyranosyl-(1→2)]-3,4-di-O-acetyl-α-D-mannopyranoside

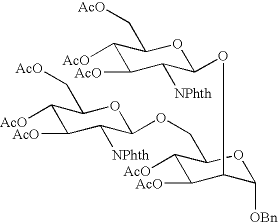

Compound 4 (10.5 g), compound 2 (1.5 g), 2,6-di-tert-butyl-4-methylpyridine (5.2 g) and activated powdered molecular sieves (˜10 g) were added to dry dichloromethane and the mixture stirred for 1 hour under argon. Methyl triflate (2.1 mL) was then added. After 17 hours methyl triflate (0.34 mL) was added and after a further 2.5 hours more methyl triflate was added (0.25 mL). After a total of 40 hours triethylamine (4 mL) was added and the mixture was filtered (Celite), evaporated under reduced pressure and dissolved in ethyl acetate (400 mL). The resulting solution was washed with water (2×40 mL) and brine (80 mL), dried (MgSO₄) and evaporated under reduced pressure. Chromatography [SiO₂, ethyl acetate:petrol (50:50) to (67:33)] gave a compound (4.1 g) which was dissolved in a mixture of trifluoroacetic acid (45 mL) and water (5 mL). After 2 minutes the mixture was concentrated under reduced pressure and dichloromethane (75 mL) and saturated sodium hydrogen carbonate (75 mL) were added. The organic phase was separated and the aqueous phase extracted with dichloromethane (2×75 mL). The combined organic fractions were dried (MgSO₄), evaporated under reduced pressure and chromatographed [SiO₂, ethyl acetate:petrol (80:20) to (90:10)] to give a compound (1.84 g) which was dissolved in dry pyridine (50 mL) and cooled to 0° C. Acetic anhydride (25 mL) was added dropwise to the stirred mixture and the reaction was allowed to warm to room temperature. After 18 hours the mixture was cooled to 0° C., water (100 mL) and ethyl acetate (300 mL) were added and the organic phase separated. The aqueous phase was extracted with ethyl acetate and the combined organic fractions were washed with 2M hydrochloric acid (2×125 mL), saturated sodium hydrogen carbonate (125 mL) and brine (125 mL), dried (MgSO₄) and evaporated under reduced pressure. Chromatography [SiO₂, ethyl acetate:petrol (67:33) to (75:25)] gave compound 5 (3.3 g).

### Compound 6

### 3,4,6-Tri-O-acetyl-2-deoxy-2-phthalimido-β-D-glucopyranosyl-(1→6)-[3,4,6-tri-O-acetyl-2-deoxy-2-phthalimido-β-D-glucopyranosyl-(1→2)]-3,4-di-O-acetyl-D-mannopyranose

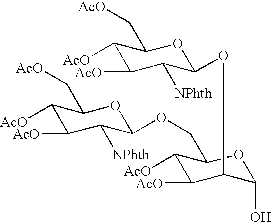

Palladium hydroxide (1.5 g, 20% on carbon) was added to a stirred solution of compound 5 (1.7 g) in ethanol (250 mL). The mixture was purged with hydrogen and stirred under an atmosphere of hydrogen for 2 days. The mixture was then filtered (Celite) and evaporated under reduced pressure to give compound 6 (1.39 g).

### Compound 7

### 3,4,6-Tri-O-acetyl-2-deoxy-2-phthalimido-β-D-glucopyranosyl-(1→6)-[3,4,6-tri-O-acetyl-2-deoxy-2-phthalimido-β-D-glucopyranosyl-(1→2)]-3,4-di-O-acetyl-α-D-mannopyranosyl trichloroacetimidate

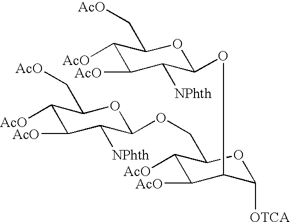

Trichloroacetonitrile (0.32 mL) and 1,8-diazabicyclo[5.4.0]undec-7-ene (0.005 mL) were added to a stirred mixture of compound 6 (0.35 g) and activated powdered molecular sieves (˜0.3 g) in dichloromethane (370 mL) at room temperature under argon. After 1 hour trichloroacetonitrile (0.2 mL) was added. After 3 hours the mixture was filtered (Celite), evaporated under reduced pressure and chromatographed (loading on the column in dichloromethane) [SiO₂, ethyl acetate:petrol:triethylamine (60:40:1)] to give compound 7 (0.36 g).

### Compound 8

### 4′-Pentenyl α-D-mannopyranose

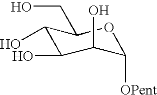

Camphorsulfonic acid (0.2 g) was added to stirred mixture of D-mannose (15 g) and 4-penten-1-ol (100 g) and the mixture heated to 100° C. After 20 hours the mixture was evaporated under reduced pressure and the residue chromatographed [SiO₂, ethyl acetate] to give compound 8 (17.1 g).

### Compound 9

### 4′-Pentenyl 3,6-di-O-benzyl-α-D-mannopyranose

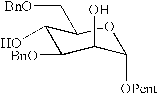

Bis[tri-n-butyltin(IV)]oxide (61 g) was added to a stirred mixture of compound 8 (16.9 g) in toluene (500 mL). The mixture was heated to 90° C. until the reagents dissolved. The apparatus was then fitted with a Dean-Stark condenser and the mixture heated at 145° C. for 4 hours. The mixture was cooled to room temperature, evaporated under reduced pressure and benzyl bromide (100 mL) added. The mixture was heated at 90° C. for 20 hours, cooled to room temperature, evaporated under reduced pressure and chromatographed [SiO₂, ethyl acetate:petrol (20:80) to (33:67)] to give compound 9 (23 g).

### Compound 10

### 3,4,6-Tri-O-acetyl-2-deoxy-2-phthalimido-D-glucopyranose

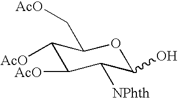

Benzylamine (12.8 mL) was added to a stirred solution of compound 3 (50.8 g) in tetrahydrofuran (320 mL). After 24 hours 1M hydrochloric acid (22 mL) was added and the mixture stirred for 5 minutes. Dichloromethane (1 L) and 1M hydrochloric acid (200 mL) were added and the organic phase separated. The aqueous phase was extracted with dichloromethane (3×300 mL) and the combined extracts dried (MgSO₄), evaporated under reduced pressure and chromatographed [SiO₂, dichloromethane:ethyl acetate (50:50)] to give compound 10 (20.8 g).

### Compound 11

### 3,4,6-Tri-O-acetyl-2-deoxy-2-Phthalimido-D-glucopyranosyl trichloroacetimidate

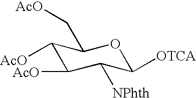

Trichloroacetonitrile (12.2 mL) and 1,8-diazabicyclo[5.4.0]undec-7-ene (0.037 mL) were added to a stirred solution of compound 10 (5.5 g) in dichloromethane (35 mL) under argon. After 30 minutes 1,8-diazabicyclo[5.4.0]undec-7-ene (0.037 mL) was added. After 1 hour the mixture was evaporated under reduced pressure and chromatographed [SiO₂, ethyl acetate:petrol:triethylamine (40:60:1)] to give compound 11 (6.01 g).

### Compound 12

### 4′-Pentenyl 3,4,6-tri-O-acetyl-2-deoxy-2-phthalimido-β-D-glucopyranosyl-(1→4)-[3,4,6-tri-O-acetyl-2-deoxy-2-phthalimido-β-D-glucopyranosyl-(1→2)]-3,6-di-O-benzyl-α-D-mannopyranoside

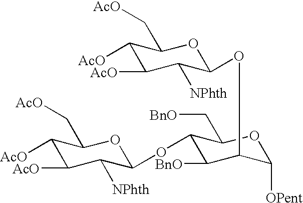

A mixture of compound 9 (2.57 g), compound 11 (9.54 g) and activated powdered molecular sieves (3.5 g) were stirred in dichloromethane (100 mL) at 0° C. for 10 minutes and room temperature for 20 minutes. Trimethylsilyl triflate (0.108 mL) was added and after 1.5 hours triethylamine (0.8 mL) was added. The mixture was filtered (Celite), evaporated under reduced pressure and chromatographed [SiO₂, ethyl acetate:petrol (40:60) to (45:55)] to give compound 12 (6.56 g).

### Compound 13

### Para-MethoxyPhenyl 3,4,6-tri-O-acetyl-2-deoxy-2-phthalimido-β-D-glucopyranoside

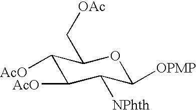

Boron trifluoride etherate (40.4 mL) was added to a stirred solution of compound 3 (76.9 g) and para-methoxyphenol (50 g) in dichloromethane (950 mL) at 0° C. After 1 hour the mixture was allowed to warm to room temperature. After 24 hours the mixture was washed successively with water (×2), 1M sodium hydroxide (×2), water (×2) and brine, dried (MgSO₄) and evaporated under reduced pressure. Crystallisation from ethyl acetate-petrol gave compound 13 (62.5 g).

### Compound 14

### para-Methoxyphenyl 4,6-O-benzylidene-2-deoxy-2-phthalimido-β-D-glucopyranoside

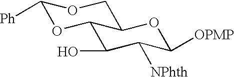

Sodium methoxide (0.5 g) was added to a stirred mixture of compound 13 (33.2 g) in methanol (450 mL). After 20 hours Dowex 50WX2 (˜2 spatulas) was added. After 30 minutes the solution was neutral. Methanol (100 mL) was added and the mixture warmed to dissolve the precipitate, filtered and evaporated under reduced pressure. Toluene (50 mL) was added and the mixture evaporated under reduced pressure. Acetonitrile (350 mL), benzaldehyde dimethyl acetal (20.2 mL) and para-toluenesulfonic acid (2.41 g) were added. After 20 hours triethylamine (4.7 mL) was added and the mixture evaporated under reduced pressure and crystallised from methanol to give compound 14 (25.5 g).

### Compound 15

### para-Methoxyphenyl 3-O-benzyl-4,6-O-benzylidene-2-deoxy-2-phthalimido-β-D-glucopyranoside

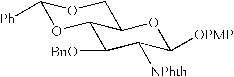

Sodium hydride (1.78 g of a 60% suspension in oil) was added to a stirred solution of compound 14 (15 g) in N,N-dimethylformamide (80 mL) at 0° C. under argon. After 15 minutes benzyl bromide (7.1 mL) was added and the mixture allowed to warm to room temperature. After 6 hours sodium hydride (1.0 g of a 60% suspension in oil) and benzyl bromide (4.0 mL) were added. After 24 hours methanol (10 mL) was added and the mixture evaporated under reduced pressure. The product was dissolved in ethyl acetate and washed with brine (×3), dried (MgSO₄) and evaporated under reduced pressure. Chromatography [SiO₂, ethyl acetate:petrol (30:70) to (40:60)] gave a solid. This was recrystallised from ethyl acetate-petrol to give compound 15 (12 g). A second crop of compound 15 was taken (1.8 g).

### Compound 16

### para-MethoxyPhenyl 3-O-benzyl-2-deoxy-2-phthalimido-β-D-glucopyranoside

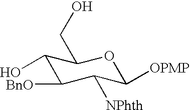

para-Toluenesulfonic acid (0.6 g) was added to a stirred solution of compound 15 (13.0 g) in methanol (260 mL) and 1,4-dioxane (145 mL). The mixture was refluxed for 1.5 hours and then cooled to room temperature and evaporated under reduced pressure. Chromatography [SiO₂, ethyl acetate:petrol (60:40)] gave compound 16 (10.48 g).

### Compound 17

### para-Methoxyphenyl 3-O-benzyl-2-deoxy-2-phthalimido-6-O-tert-butyldimethylsilyl-β-D-glucopyranoside

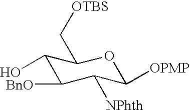

Imidazole (3.5 g) and tert-butyldimethylsilyl chloride (3.81 g) were added to a stirred mixture of compound 16 (10.4 g) in anhydrous N,N-dimethylformamide at 0° C. under argon. After 2 hours the mixture was evaporated under reduced pressure and ethyl acetate added to dissolve the product. The mixture was washed with water (×2) and brine (×2), dried (MgSO₄) and evaporated under reduced pressure. Chromatography [SiO₂, ethyl acetate:petrol (30:70)] gave compound 17 (12.43 g).

### Compound 18

### para-Methoxyphenyl 3,6-di-O-benzyl-2-deoxy-2-phthalimido-β-D-glucopyranoside

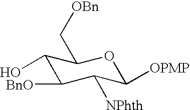

Compound 14 (22.7 g) was added to a stirred mixture of activated powdered molecular sieves (˜5 g) in tetrahydrofuran (800 mL) at 0° C. under argon. After 1 hour sodium cyanoborohydride (50 g) and methyl orange (1 speck) were added. 4M Hydrogen chloride in 1,4-dioxane was added as rapidly as the effervescence allowed until a permanent pink colour developed (˜170 mL). After 24 hours the mixture was poured into ice-water and then filtered (Celite). The mixture was extracted with dichloromethane (×2) and the combined extracts stirred with 2M hydrochloric acid (˜200 mL) for 24 hours. The organic phase was then separated and washed with saturated sodium hydrogen carbonate (×2) and water, dried (MgSO₄) and evaporated under reduced pressure. Chromatography [SiO₂, ethyl acetate:petrol (30:70)] gave compound 18 (21.2 g).

### Compound 19

### para-Methoxyphenyl 3,6-di-O-benzyl-4-O-levulinyl-2-deoxy-2-phthalimido-β-D-glucopyranoside

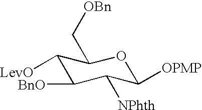

N,N-Dicyclohexylcarbodiimide (11.8 g) was added to a solution of levulinic acid (13.3 g) in dichloromethane (80 mL) at 0° C. After 10 minutes the mixture was allowed to warm to room temperature and stirred for 3 hours. The mixture was then filtered into a solution of compound 18 (6.8 g) in pyridine (70 mL), washing through with dichloromethane (20 mL). After 3 days the mixture was poured into ice-water and stirred for 30 minutes. The mixture was extracted with dichloromethane and the extracts washed with 2M hydrochloric acid (×2), saturated sodium hydrogen carbonate (×2) and water, dried (MgSO₄) and evaporated under reduced pressure. Chromatography [SiO₂, ethyl acetate:petrol (40:60) to (50:50)] gave compound 19 (7.83 g).

### Compound 20

### 3,6-Di-O-benzyl-4-O-levulinyl-2-deoxy-2-phthalimido-D-glucopyranose

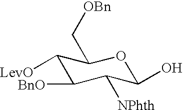

Ceric ammonium nitrate (28.1 g) was added to a vigorously stirred mixture of compound 19 (7.82 g) in toluene (115 mL), acetonitrile (84 mL) and water (37 mL). After 2 hours ethyl acetate was added and the mixture washed with water (×2) and the combined aqueous extracts re-extracted with ethyl acetate. The combined organic fractions were washed with saturated sodium hydrogen carbonate and brine, dried (MgSO₄) and evaporated under reduced pressure to give compound 20 (6.64 g).

### Compound 21

### 3,6-Di-O-benzyl-4-O-levulinyl-2-deoxy-2-phthalimido-β-D-glucopyranosyl Trichloroacetimidate

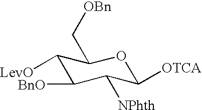

Trichloroacetonitrile (11.3 mL) was added to a stirred mixture of compound 20 (6.64 g) and activated powdered molecular sieves (˜2 g) in dichloromethane (71 mL). After 2 hours 1,8-diazabicyclo[5.4.0]undec-7-ene (0.56 mL) was added. After 1 hour the mixture was filtered (Celite) and evaporated under reduced pressure. Chromatography [SiO₂, ethyl acetate:petrol:triethylamine (44:55:1)] gave compound 21 (5.63 g).

### Compound 22

### para-Methoxyphenyl 3,6-di-O-benzyl-4-O-levulinyl-2-deoxy-2-phthalimido-β-D-glucopyranosyl-(1→4)-3-O-benzyl-2-deoxy-2-phthalimido-6-O-tert-butyldimethylsilyl-β-D-glucopyranoside

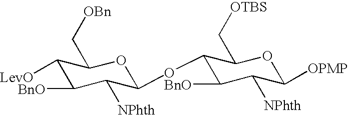

Compound 21 (2.51 g), compound 18 (1.66 g) and powdered activated molecular sieves (˜1 g) were stirred in dichloromethane (75 mL). After 20 minutes the mixture was cooled to −78° C. and boron trifluoride etherate (0.32 mL) added. After 5 hours at −78° C. the mixture was allowed to slowly warm to 0° C. overnight. The mixture was then warmed to room temperature and filtered (Celite) washing through with dichloromethane (100 mL). The mixture was washed with saturated sodium hydrogen carbonate and brine, dried (MgSO₄), evaporated under reduced pressure and chromatographed [SiO₂, ethyl acetate:petrol (30:70)] to give compound 22 (2.02 g).

### Compound 23

### para-Methoxyphenyl 3,6-di-O-benzyl-4-O-levulinyl-2-deoxy-2-phthalimido-β-D-glucopyranosyl-(1→4)-3-O-benzyl-2-deoxy-2-phthalimido-β-D-glucopyranoside

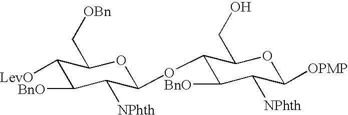

para-Toluenesulfonic acid was added to a stirred mixture of compound 22 (2.0 g) in acetonitrile (21.5 mL) and water (1.2 mL) to adjust the pH to 3. After 3.5 hours ethyl acetate was added and the mixture washed with water, saturated sodium hydrogen carbonate and brine, dried (MgSO₄), evaporated under reduced pressure and chromatographed [SiO₂, ethyl acetate:petrol (50:50) to (75:25)] to give compound 23 (1.49 g).

### Compound 24

### 1,2,3,4-Tetra-O-acetyl-L-fucopyranose

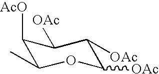

Acetic anhydride (57 mL) was added over 10 minutes to a stirred solution of L-(−)-fucose (8.2 g) in pyridine (80 mL) at 0° C. After 2 hours the mixture was warmed to room temperature. After 20 hours the mixture was evaporated under reduced pressure and water (200 mL) added. The mixture was extracted with dichloromethane (×4) and the combined extracts were washed with 1M hydrochloric acid (×2), saturated sodium hydrogen carbonate (×2) and water, dried (MgSO₄) and evaporated under reduced pressure to give compound 24 (15.6 g).

### Compound 25

### Phenyl 2,3,4-tri-O-acetyl-1-thio-β-L-fucopyranoside

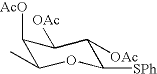

Thiophenol (10.5 mL) and boron trifluoride etherate (16.3 mL) were added to a stirred solution of compound 24 (15.6 g) in dichloromethane (160 mL) at 0° C. After 10 minutes the mixture was warmed to room temperature. After 2 hours dichloromethane (80 mL) was added and the mixture washed with 1M sodium carbonate (×2) and water, dried (MgSO₄) and evaporated under reduced pressure. Chromatography [SiO₂, ethyl acetate:petrol (20:80)] gave compound 25 (14.8 g).

### Compound 26

### Phenyl 1-thio-β-L-fucopyranoside

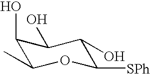

Sodium methoxide (0.12 g) was added to a stirred solution of compound 25 (14.8 g) in methanol (70 mL) under argon. After 18 hours Dowex 50×2 (1 spatula) was added. After 30 minutes the mixture was filtered and evaporated under reduced pressure to give compound 26 (8.9 g).

### Compound 27

### Phenyl 2,3,4-tri-O-benzyl-1-thio-β-L-fucopyranoside

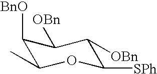

Sodium hydride (10.6 g of a 60% suspension in oil) was added to a stirred solution of compound 26 (8.9 g) in N,N-dimethylformamide (140 mL) at 0° C. After 15 minutes when the evolution of hydrogen had ceased benzyl bromide (17.3 mL) was added. The mixture was warmed to room temperature and after 2 hours sodium hydride (0.5 g of a 60% suspension in oil) was added. After 2 hours water (10 mL) was added slowly and the mixture added to ethyl acetate (500 mL). The mixture was washed with water (×3) and brine. Petrol was added and the mixture was dried (MgSO₄) and evaporated under reduced pressure. Chromatography [SiO₂, ethyl acetate:petrol (20:80) to (50:50)] gave a solid product that was recrystallised from ethyl acetate-petrol to give compound 27 (11.7 g).

### Compound 28

### para-Methoxyphenyl 3,6-di-O-benzyl-4-O-levulinyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranoside

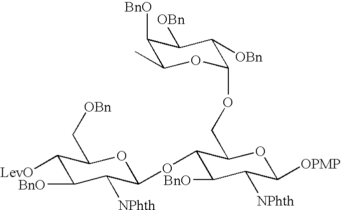

Compound 23 (1.0 g), compound 28 (0.486 g) and powdered activated molecular sieves (˜1 g) were stirred in diethyl ether (47 mL) under argon for 30 minutes. The mixture was then cooled to −78° C. and N-iodosuccinimide (0.532 g) and silver triflate (0.255 g) were added. After stirring at −78° C. overnight the mixture was allowed to warm to room temperature, dichloromethane was added and the mixture was filtered (Celite), washed with 10% sodium thiosulfate and brine, dried (MgSO₄) and evaporated under reduced pressure. Chromatography [SiO₂, ethyl acetate:petrol (40:60)] gave compound 28 (0.874 g).

### Compound 29

### para-MethoxyPhenyl 3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranoside

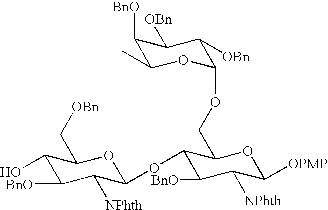

Hydrazine monohydrate (1.35 mL) was added to a stirred solution of compound 28 (4.03 g) in pyridine (76.5 mL) and acetic acid (19.8 mL). After 50 minutes ethyl acetate was added and the mixture washed with saturated sodium hydrogen carbonate (×3) and brine. The aqueous washings were extracted with ethyl acetate. The combined organic fractions were washed with saturated sodium hydrogen carbonate and brine, dried (MgSO₄), evaporated under reduced pressure and chromatographed [SiO₂, ethyl acetate:petrol (30:70) to (50:50)] to give compound 29 (0.266 g).

### Assembly

### Compound 30

### Ethyl 2,3,4,6-tetra-O-acetyl-1-thio-β-D-glucopyranose

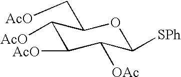

Ethanethiol (10.3 g) was added to a stirred solution of glucose pentaacetate (50 g) in dry dichloromethane (100 mL). The mixture was cooled to 0° C. and boron trifluoride etherate (19.5 mL) was added dropwise. After 8 hours the mixture was poured into cooled saturated sodium hydrogen carbonate (250 mL) and the organic phase was separated. The aqueous phase was extracted with dichloromethane (2×50 mL). The combined organic extracts were washed with water and brine, dried (MgSO₄), evaporated under reduced pressure and crystallised from petrol-dichloromethane (2 crops) to give compound 30 (42.8 g).

### Compound 31

### Ethyl 1-thio-β-D-glucopyranose

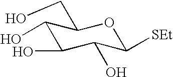

Sodium methoxide (0.275 g) was added to a stirred solution of compound 30 (20 g) in methanol (125 mL). After 4 hours Dowex 50×2 was added to neutralise the mixture and the mixture was filtered and evaporated under reduced pressure to give compound 31 (11.1 g).

### Compound 32

### Ethyl 4,6-O-benzylidene-1-thio-β-D-glucopyranose

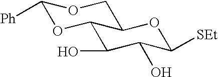

Benzaldehyde dimethyl acetal (14.9 g) and camphorsulfonic acid (1.13 g) were added to a mixture of compound 31 (11 g) in N,N-dimethylformamide (50 mL). The mixture was heated at 60° C. at 189 mbar for 4 hours. The mixture was then cooled to room temperature and triethylamine added to make the mixture basic. The mixture was evaporated under reduced pressure and dissolved in ethyl acetate (200 mL). The mixture was washed with saturated sodium hydrogen carbonate, water and brine, dried (MgSO₄) and evaporated under reduced pressure. Crystallisation from ethyl acetate-petrol gave compound 32 (10.5 g).

### Compound 33

### Ethyl 4,6-O-benzylidene-3-O-tert-butyldimethylsilyl-1-thio-β-D-glucopyranose

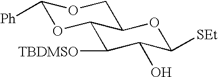

Imidazole (1.96 g) was added to a stirred solution of compound 32 (6 g) and tert-butyldimethylsilyl chloride (3.46 g) in N,N-dimethylformamide (100 mL) at 0° C. The mixture was warmed to room temperature and stirred overnight. Ethyl acetate was added and the mixture was thoroughly washed with water, dried (MgSO₄) and evaporated under reduced pressure. Chromatography [SiO₂, ethyl acetate:petrol (40:60)] gave compound 33 (8.07 g).

### Compound 34

### Ethyl 4,6-O-benzylidene-2-O-levulinyl-3-O-tert-butyldimethylsilyl-1-thio-β-D-glucopyranose

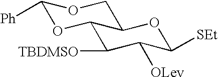

N,N-Dicyclohexylcarbodiimide (7.78 g) and 4-dimethylaminopyridine (2.52 g) were added to a stirred solution of compound 33 (8.05 g) in dichloromethane (150 mL) at 0° C. After 10 minutes levulinic acid (4.37 g) in dichloromethane (50 mL) was added dropwise and the mixture warmed to room temperature. After 5 hours the mixture was filtered and the filtrate washed with saturated sodium hydrogen carbonate and water, dried (MgSO₄) and evaporated under reduced pressure. Chromatography [SiO₂, ethyl acetate:petrol (15:85)] gave compound 34 (8.9 g).

### Compound 35

### para-MethoxyPhenyl 4,6-O-benzylidene-2-O-levulinyl-3-O-tert-butyldimethylsilyl-β-D-glucopyranosyl-(1→4)-3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranoside.

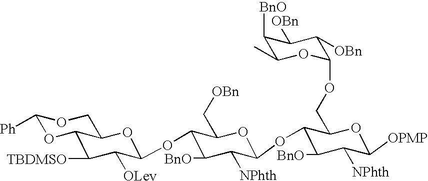

Compound 34 (1.53 g) and compound 29 (3.25 g) were placed under reduced pressure overnight. N-Iodosuccinimide (0.785 g), activated powdered molecular sieves and dichloromethane (40 mL) were then added and the mixture stirred. After 5 minutes the mixture was cooled to 0° C. and trimethylsilyl triflate (0.53 mL) was added. After 30 minutes the mixture was warmed to room temperature over 30 minutes. The mixture was filtered (Celite) and the filtrate washed with 10% sodium thiosulfate, saturated sodium hydrogen carbonate and brine, dried (MgSO₄) and evaporated under reduced pressure. Chromatography [SiO₂, ethyl acetate:petrol (50:50)] gave compound 35 (3.54 g).

### Compound 36

### Para-Methoxyphenyl 4,6-O-benzylidene-2-O-levulinyl-β-D-glucopyranosyl-(1→4)-3,6-di-O-benzyl-2-deoxy-2-Phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranoside

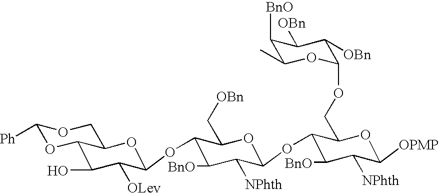

Triethylamine trihydrofluoride (6.81 mL) was added dropwise to a stirred mixture of compound 35 (3.88 g) in dry tetrahydrofuran (15 mL). After 55 hours dichloromethane was added and the mixture washed repeatedly with saturated sodium hydrogen bicarbonate and then with brine. The mixture was dried (MgSO₄), evaporated under reduced pressure and chromatographed [SiO₂, ethyl acetate:petrol (40:60) to (60:40)] to give compound 36 (3.01 g).

### Compound 37

### para-Methoxyphenyl {3,4,6-tri-O-acetyl-2-deoxy-2-phthalimido-β-D-glucopyranosyl-(1→4)-[3,4,6-tri-O-acetyl-2-deoxy-2-phthalimido-β-D-glucopyranosyl-(1→2)]-3,6-di-O-benzyl-α-D-mannopyranosyl-(1→3)}-4,6-O-benzylidene-2-O-levulinyl-β-D-glucopyranosyl-(1→4)-3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranoside

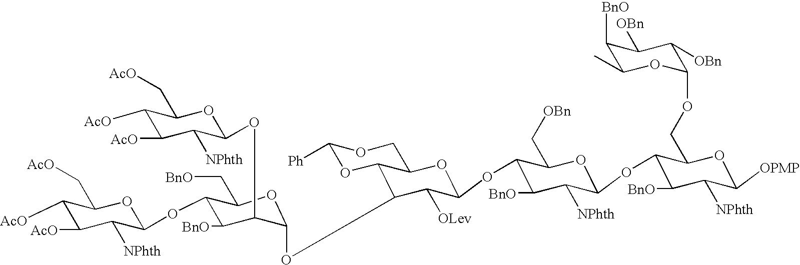

Compound 36 (2.0 g) and compound 12 (2.9 g) were placed under reduced pressure for 3 hours. N-Iodosuccinimide (1.28 g), powdered molecular sieves and dichloromethane (35 mL) were added. The mixture was stirred under argon and trimethylsilyl triflate (0.33 mL) added. After 45 minutes the mixture was filtered (Celite) and washed with 10% sodium thiosulfate, sodium hydrogen carbonate and brine, dried (MgSO₄) and evaporated under reduced pressure. Chromatography [SiO₂, ethyl acetate:petrol (50:50)] gave a product that was chromatographed [SiO₂, ethyl acetate:toluene (40:60)] to give compound 37 (2.34 g).

### Compound 38

### para-Methoxyphenyl {3,4,6-tri-O-acetyl-2-deoxy-2-phthalimido-β-D-glucopyranosyl-(1→4)-[3,4,6-tri-O-acetyl-2-deoxy-2-phthalimido-β-D-glucopyranosyl-(1→2)]-3,6-di-benzyl-α-D-mannopyranosyl-(1→3)}-4,6-O-benzylidene-β-D-glucopyranosyl-(1→4)-3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-Phthalimido-β-D-glucopyranoside

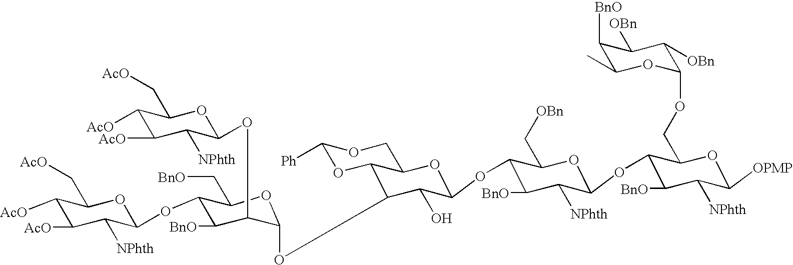

Hydrazine acetate (0.149 g) was added to a mixture of compound 37 (2.369 g) in methanol (21 mL) and tetrahydrofuran (7 mL) and the mixture stirred overnight. The mixture was evaporated under reduced pressure, dichloromethane was added and the mixture was washed with water and brine, dried (MgSO₄) and evaporated under reduced pressure. Chromatography [SiO₂, ethyl acetate:petrol (70:30)] gave compound 38 (1.84 g).

### Compound 39

### para-Methoxyphenyl {3,4,6-tri-O-acetyl-2-deoxy-2-phthalimido-β-D-glucopyranosyl-(1→4)-[3,4,6-tri-O-acetyl-2-deoxy-2-phthalimido-β-D-glucopyranosyl-(1→2)]-3,6-di-O-benzyl-α-D-mannopyranosyl-(1→3)}-4,6-O-benzylidene-2-O-acetyl-O-D-mannopyranosyl-(1→4)-3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranoside

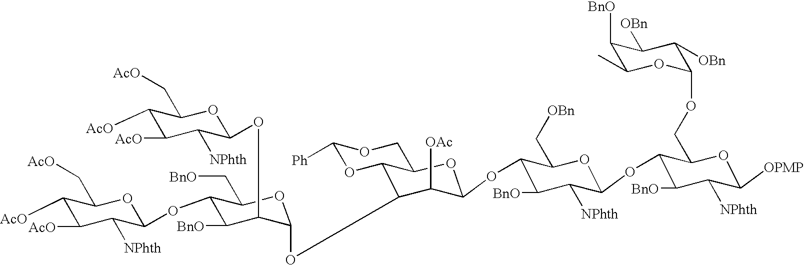

Pyridine (1.05 g) was added to compound 38 (0.75 g) in dichloromethane (15 mL) under argon. The mixture was stirred and cooled to 0° C. and triflic anhydride (1.12 g) added dropwise. After 30 minutes the mixture was warmed to room temperature and stirred for 6 hours. Dichloromethane was added and the mixture washed with saturated sodium hydrogen carbonate and dried (MgSO₄). Tetrabutylammonium acetate (1.12 g) and dry toluene (20 mL) were added and the reaction vessel placed in a sonic bath for 20 hours after which the mixture was chromatographed [SiO₂, ethyl acetate:petrol (50:50) to (60:40)] to give compound 39 (0.573 g).

### Compound 40

### para-Methoxyphenyl {3,4,6-tri-O-acetyl-2-deoxy-2-phthalimido-β-D-glucopyranosyl-(1→4)-[3,4,6-tri-O-acetyl-2-deoxy-2-phthalimido-1-D-glucopyranosyl-(1→2)]-3,6-di-O-benzyl-α-D-mannopyranosyl-(1→3)}-2-O-acetyl-β-D-mannopyranosyl-(1→4)-3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-Phthalimido-β-D-glucopyranoside

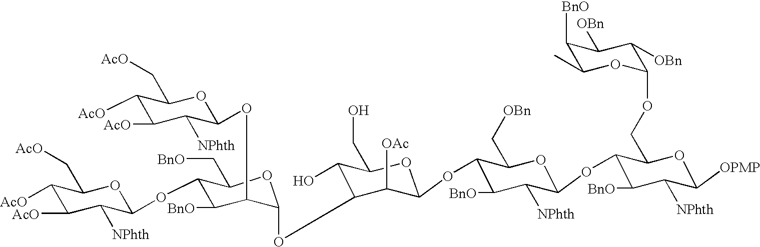

Ethanethiol (1.82 mL) was added to a stirred solution of compound 39 (1.41 g) in dichloromethane (20 mL). The mixture was cooled to 0° C. and boron trifluoride etherate (0.13 mL) in dichloromethane (1 mL) added dropwise and the mixture was warmed to room temperature. After 1 hour boron trifluoride etherate (0.07 mL) in dichloromethane (0.5 mL) was added. After 1 hour excess triethylamine was added followed by dichloromethane and the mixture washed with saturated sodium hydrogen carbonate, dried (MgSO₄) and evaporated under reduced pressure. Chromatography [SiO₂, ethyl acetate:petrol (70:30)] gave compound 40 (0.763 g).

### Compound 41

### para-Methoxyphenyl {3,4,6-tri-O-acetyl-2-deoxy-2-phthalimido-β-D-glucopyranosyl-(1→4)-[3,4,6-tri-O-acetyl-2-deoxy-2-phthalimido-1-D-glucopyranosyl-(1→2)]-3,6-di-O-benzyl-α-D-mannopyranosyl-(1→3)}-{3,4,6-tri-O-acetyl-2-deoxy-2-phthalimido-β-D-glucopyranosyl-(1→6)-[3,4,6-tri-O-acetyl-2-deoxy-2-phthalimido-β-D-glucopyranosyl-(1→2)]-3,4-di-O-acetyl-α-D-mannopyranosyl-(1→6)}-2-O-acetyl-β-D-mannopyranosyl-(1-4)-3,6-di-O-benzyl-2-deoxy-2-Phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranoside

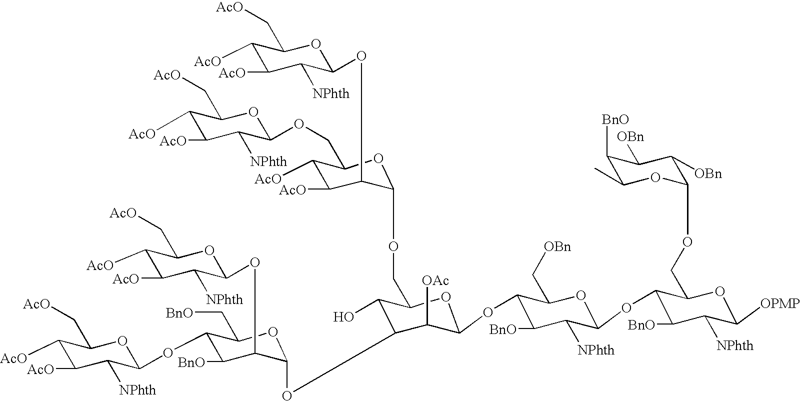

Compound 40 (0.33 g) and compound 7 (0.192 g) were placed under reduced pressure. After 5 hours activated powdered molecular sieves (0.3 g) and dichloromethane (8.5 mL) were added and the mixture stirred under argon. After 20 minutes the mixture was cooled to 0° C. and trimethylsilyl triflate (0.0043 mL) in dichloromethane (0.5 mL) was added dropwise. After 1 hour the mixture was filtered (Celite) and the filtrate washed with saturated sodium hydrogen carbonate, dried (MgSO₄) and evaporated under reduced pressure. Radial chromatography [SiO₂, ethyl acetate:petrol (75:25)] gave compound 41 (0.302 g).

### Compound 42

### para-Methoxyphenyl {3,4,6-tri-O-acetyl-2-deoxy-2-phthalimido-β-D-glucopyranosyl-(1→4)-[3,4,6-tri-O-acetyl-2-deoxy-2-phthalimido-β-D-glucopyranosyl-(1→2)]-3,6-di-O-benzyl-α-D-mannopyranosyl-(1→3)}-{3,4,6-tri-O-acetyl-2-deoxy-2-phthalimido-β-D-glucopyranosyl-(1→6)-[3,4,6-tri-O-acetyl-2-deoxy-2-phthalimido-β-D-glucopyranosyl-(1→2)]-3,4-di-O-acetyl-α-D-mannopyranosyl-(1→6)-2,4-di-O-acetyl-β-D-mannopyranosyl-(1→4)-3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranoside

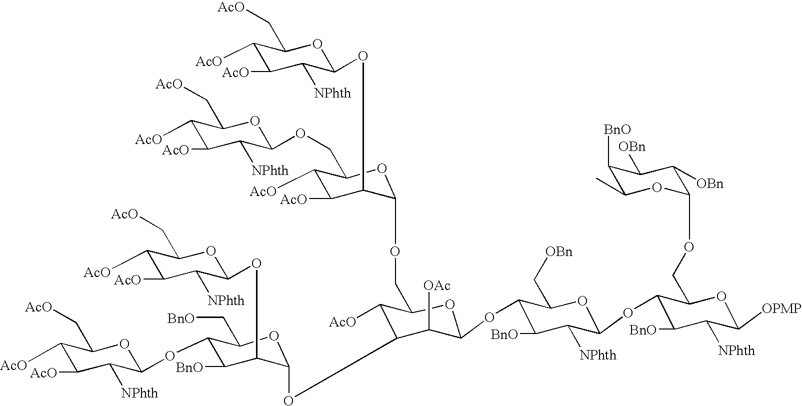

Acetic anhydride (0.75 mL) was added to a stirred solution of compound 41 (0.159 g) in pyridine (4 mL). After 20 hours ethyl acetate was added and the mixture was washed with saturated sodium hydrogen carbonate, water and brine, dried (MgSO₄) and evaporated under reduced pressure to give compound 42 (0.163 g) which was used without further purification.

### Compound 43

### {3,4,6-Tri-O-acetyl-2-deoxy-2-phthalimido-β-D-glucopyranosyl-(1→4)-[3,4,6-tri-O-acetyl-2-deoxy-2-phthalimido-β-D-glucopyranosyl-(1→2)]-3,6-di-O-benzyl-α-D-mannopyranosyl-(1→3)}-{3,4,6-tri-O-acetyl-2-deoxy-2-phthalimido-β-D-glucopyranosyl-(1→6)-[3,4,6-tri-O-acetyl-2-deoxy-2-phthalimido-β-D-glucopyranosyl-(1→2)]-3,4-di-O-acetyl-α-D-mannopyranosyl-(1→6)}-2,4-di-O-acetyl-β-D-mannopyranosyl-(1→4)-3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-phthalimido-D-glucopyranose

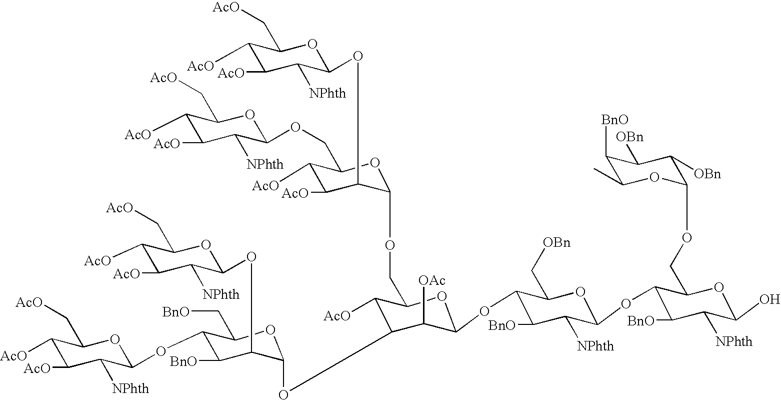

Ceric ammonium nitrate was added to a vigorously stirred mixture of compound 42 (0.163 g) in acetonitrile (2 mL), toluene (1 mL) and water (0.5 mL). After 40 minutes ethyl acetate was added and the mixture was washed with water and saturated sodium hydrogen carbonate, dried (MgSO₄) and evaporated under reduced pressure to give compound 43 (0.152 g) which was used without further purification.

### Compound 44

### {3,4,6-Tri-O-acetyl-2-deoxy-2-phthalimido-β-D-glucopyranosyl-(1→4)-[3,4,6-tri-O-acetyl-2-deoxy-2-phthalimido-β-D-glucopyranosyl-(1→2)]-3,6-di-O-benzyl-α-D-mannopyranosyl-(1→3)}-{3,4,6-tri-O-acetyl-2-deoxy-2-phthalimido-β-D-glucopyranosyl-(1→6)-[3,4,6-tri-O-acetyl-2-deoxy-2-Phthalimido-β-D-glucopyranosyl-(1→2)]-3,4-di-O-acetyl-α-D-mannopyranosyl-(1-6)}-2,4-di-O-acetyl-β-D-mannopyranosyl-(1→4)-3,6-di-O-benzyl-2-deoxy-2-Phthalimido-β-D-glucopyranosyl-(1→4)-[2,3,4-tri-O-benzyl-α-L-fucopyranosyl-(1→6)]-3-O-benzyl-2-deoxy-2-phthalimido-β-D-glucopyranosyl trichloroacetimidate

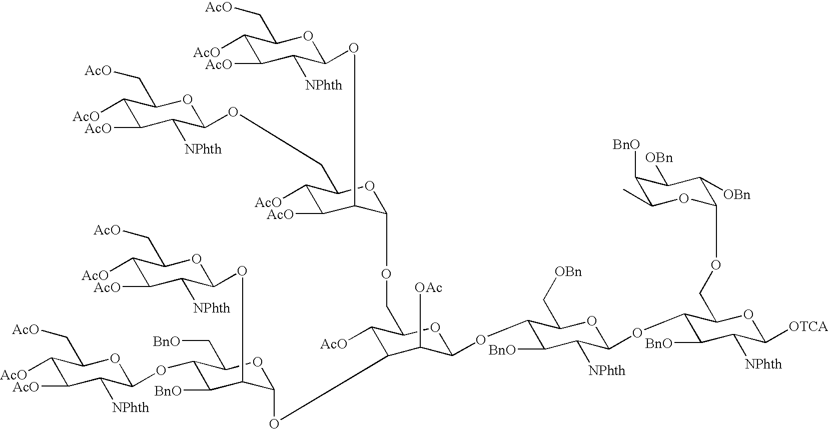

Trichloroacetonitrile (0.40 mL) was added to a stirred solution of compound 43 (0.150 g) in dichloromethane (1 mL) under argon. The mixture was cooled to 0° C. and 1,8-diazabicyclo[5.4.0]undec-7-ene (0.00176 mL) added. The mixture was allowed to warm to room temperature. After 2.5 hours the mixture was chromatographed [SiO₂, ethyl acetate:petrol:triethylamine (75:25:2)] to give compound 44 (0.122 g).

### Compound 45

### Benzyl {3,4,6-tri-O-acetyl-2-deoxy-2-phthalimido-β-D-glucopyranosyl-(1→4)-[3,4,6-tri-O-acetyl-2-deoxy-2-phthalimido-X-D-glucopyranosyl-(1→2)]-3,6-di-O-benzyl-α-D-mannopyranosyl-(1→3)}-{3,4,6-tri-O-acetyl-2-deoxy-2-phthalimido-β-D-glucopyranosyl-(1→6)-[3,4,6-tri-O-acetyl-2-deoxy-2-phthalimido-β-D-glucopyranosyl-(1→2)]-3,4-di-O-acetyl-α-D-mannopyranosyl-(1→6)}-2,4-di-O-acetyl-β-D-mannopyranosyl-(1→4)-3,6-di-O-benzyl-2-deoxy-2-phthalimido-β-D-glucopyranosyl-(1→4)-[2,3,4-tri-O-benzyl-α-L-fucopyranosyl-(1→6)]-3-O-benzyl-2-deoxy-2-phthalimido-1-thio-β-D-glucopyranoside

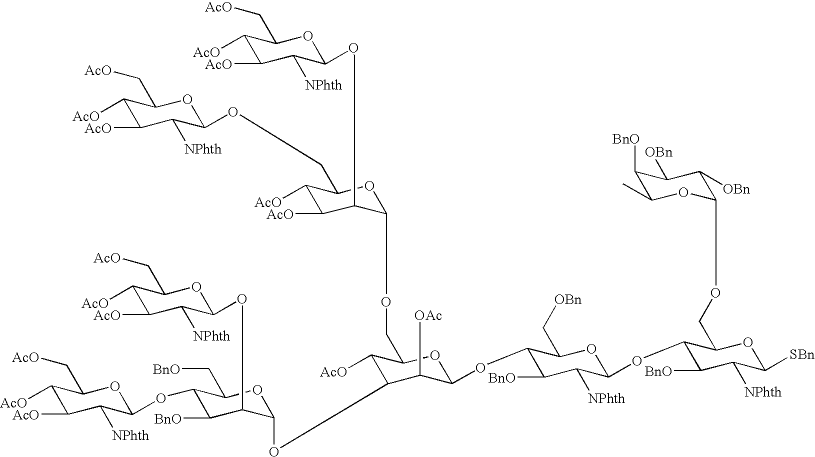

Benzyl mercaptan (5.6 mg) was added to a stirred mixture of compound 44 (0.118 g) and activated powdered molecular sieves in dichloromethane (1 mL) under argon. After 5 minutes the mixture was cooled to 0° C. and trimethylsilyl triflate (0.05 mL of a solution of 0.02 mL of trimethylsilyl triflate in 1 mL dichloromethane) added. After 40 minutes trimethylsilyl triflate (0.025 mL of a solution of 0.02 mL in 1 mL dichloromethane) was added. After 2 hours trimethylsilyl triflate (0.012 mL of a solution of 0.02 mL in 1 mL dichloromethane) was added. After 30 minutes trimethylsilyl triflate (0.012 mL of a solution of 0.02 mL in 1 mL dichloromethane) added. After 30 minutes excess triethylamine followed by ethyl acetate were added and the mixture filtered (Celite). The filtrate was washed with saturated sodium hydrogen carbonate, dried (MgSO₄) and evaporated under reduced pressure. Chromatography [SiO₂, ethyl acetate:petrol (70:30)] and radial chromatography [SiO₂, ethyl acetate:petrol (65:35)] gave compound 45 (0.042 g).

### Compound 46

### Benzyl {3,4,6-tri-O-acetyl-2-deoxy-2-acetamido-β-D-glucopyranosyl-(1→4)-[3,4,6-tri-O-acetyl-2-deoxy-2-acetamido-β-D-glucopyranosyl-(1→2)]-3,6-di-O-benzyl-α-D-mannopyranosyl-(1→3)}-{3,4,6-tri-O-acetyl-2-deoxy-2-acetamido-β-D-glucopyranosyl-(1→6)-[3,4,6-tri-O-acetyl-2-deoxy-2-acetamido-β-D-glucopyranosyl-(1→2)]-3,4-di-O-acetyl-α-D-mannopyranosyl-(1→6)}-2,4-di-O-acetyl-β-D-mannopyranosyl-(1→4)-3,6-di-O-benzyl-2-deoxy-2-acetamido-β-D-glucopyranosyl-(1→4)-[2,3,4-tri-O-benzyl-α-L-fucopyranosyl-(1→6)]-3-O-benzyl-2-deoxy-2-acetamido-1-thio-β-D-glucopyranoside

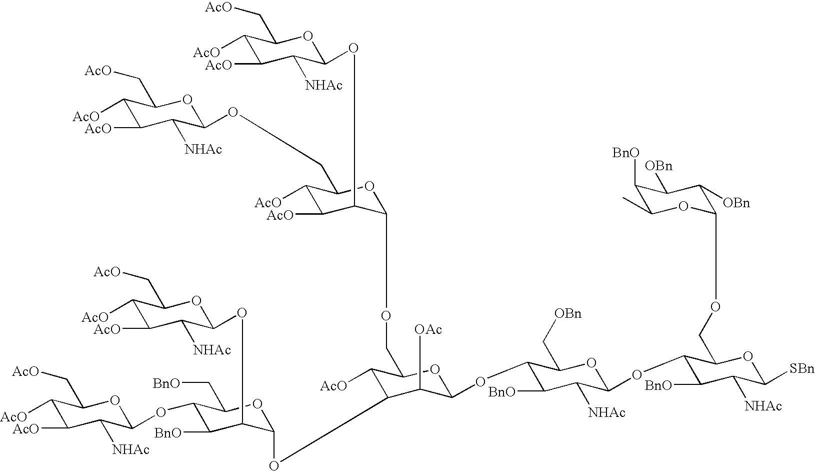

Ethylene diamine (1 mL) was added to a stirred mixture of compound 45 (0.041 g) in n-butanol (4 mL) and heated at 85° C. for 18 hours. The mixture was evaporated under reduced pressure, toluene added and evaporated under reduced pressure. Pyridine (5 mL) was added followed by acetic anhydride (1 mL). After stirring overnight under argon ethyl acetate (150 mL) was added and the mixture washed with water (10 mL). The mixture was dried (MgSO₄), evaporated under reduced pressure and radial chromatographed [SiO₂, ethyl acetate] to give compound 46 (0.024 g).

### Compound 47

### {2-Deoxy-2-acetamido-β-D-glucopyranosyl-(1→4)-[2-deoxy-2-acetamido-β-D-glucopyranosyl-(1→2)]-α-D-mannopyranosyl-(1→3)}-{2-deoxy-2-acetamido-β-D-glucopyranosyl-(1→6)-[2-deoxy-2-acetamido-β-D-glucopyranosyl-(1→2)]-α-D-mannopyranosyl-(1→6)}-β-D-mannopyranosyl-(1→4)-2-deoxy-2-acetamido-β-D-glucopyranosyl-(1→4)-[α-L-glucopyranosyl-(1→6)]-2-deoxy-2-acetamidomido-1-thio-β-D-glucopyranose

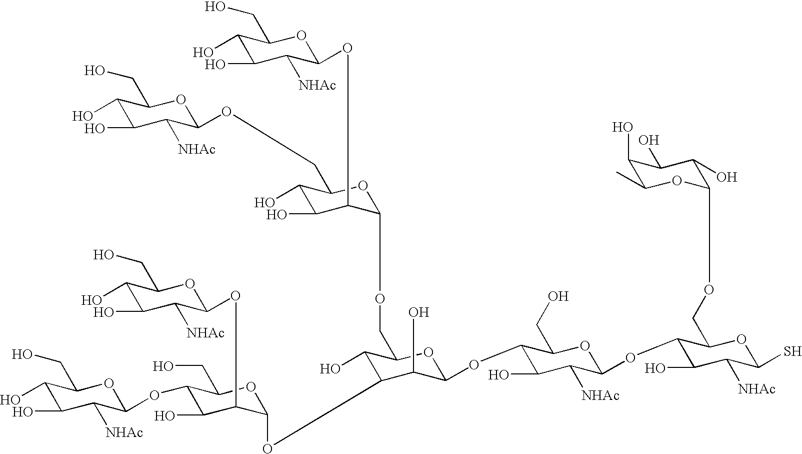

Sodium (0.005 g) was added to liquid ammonia (10 mL) at −78° C. After 10 minutes compound 46 (0.024 g) in tetrahydrofuran (2 mL) was added. After 20 minutes ammonium chloride (0.019 g) was added and the mixture allowed to warm to room temperature. Size exclusion chromatography [Biorad P2 gel, 0.04M ammonium carbonate] gave compound 47 (0.008 g).

### Compound 48

### Disulfide of 2-Deoxy-2-acetamido-β-D-glucopyranosyl-(1→4)-[2-deoxy-2-acetamido-β-D-glucopyranosyl-(1→2)]-α-D-mannopyranosyl-(1→3)}-{2-deoxy-2-acetamido-β-D-glucopyranosyl-(1→6)-[2-deoxy-2-acetamido-β-D-glucopyranosyl-(1→2)]-α-D-mannopyranosyl-(1→6)}-β-D-mannopyranosyl-(1→4)-2-deoxy-2-acetamido-β-D-glucopyranosyl-(1→4)-[α-L-fucopyranosyl-(1→6)]-2-deoxy-2-acetamidomido-1-thio-β-D-glucopyranose

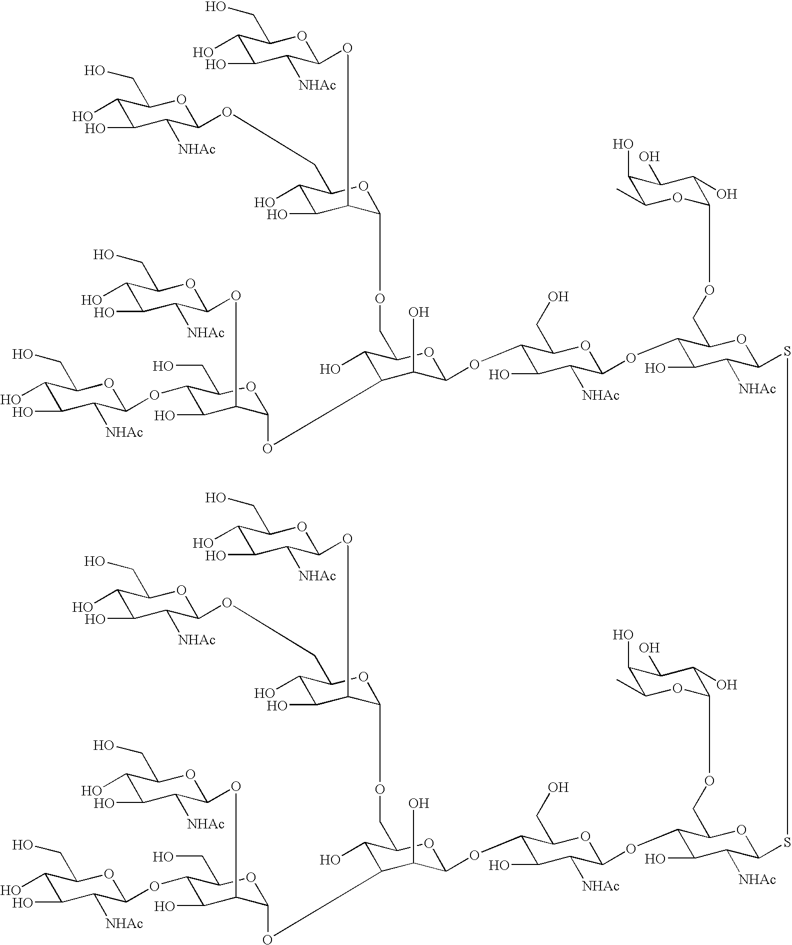

Compound 47 in water was allowed to stand under an air atmosphere until the thiol was completely converted to the disulfide (about 5 days).

### SBL-Compound 47 Conjugate

Compound 48 (20 μL of a 10 mg/mL solution) and ammonium carbonate buffer (pH 8.6) (50 μL) were added to Subtilisin Bacillus lentus (SBL) S156C mutant (50 μL of a solution of 0.4 mg in 100 μL of 50 mM ammonium carbonate buffer (pH 8.6)). After mixing for 45 minutes, a 5 UL aliquot was taken and analysed by mass spectrometry (ESI-TOF), showing conversion to the SBL-compound 47 conjugate (observed mass 28600, theoretical 28598).

SBL-Compound 47 Conjugate (Inactivated with PMSF)

Phenylmethylsulfonyl fluoride (PMSF) (3 μL of a 1.0 M solution in ethanol) and ammonium carbonate buffer (pH 8.6) (50 μL) were added to Subtilisin Bacillus lentus (SBL) S156C mutant (50 μL of a solution of 0.4 mg in 100 μL of 50 mM ammonium carbonate buffer (pH 8.6)). After 5 minutes the mixture was desalted on a Zeba Desalt Spin Column (Pierce) that had been pre-equilibrated with 50 mM ammonium carbonate buffer (pH 8.6). Compound 48 (20 μL of a 10 mg/mL solution) was added. After mixing for 2 hours a 5 μL aliquot was taken and analysed by mass spectrometry (ESI-TOF), showing conversion to the SBL-compound 47 conjugate (inactivated with PMSF) (observed mass 28752, theoretical. 28752).

### Compound 49

### Bromo-2,3,4,6-tetra-O-acetyl-α-D-mannopyranoside

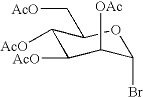

33% Hydrogen bromide in acetic acid (80 mL) was added to a stirred mixture of mannose pentaacetate (10 g) in dry dichloromethane (50 mL). After 4 hours dichloromethane (100 mL) and water (100 mL) were added and the organic phase separated and the aqueous phase was extracted with dichloromethane (2×25 mL). The combined organic fractions were washed with saturated sodium hydrogen carbonate, water and brine, dried (MgSO₄) and evaporated under reduced pressure to give compound 49 (8.06 g) which was used without further purification.

### Compound 50

### 1,2-O-(exo-1-Methoxyethylidene)-3,4,6-tri-O-acetyl-β-D-mannopyranose

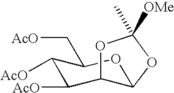

Methanol (1.58 mL) and 2,6-lutidine (4.53 mL) were added to a stirred solution of compound 49 (8.0 g) in dry dichloromethane (40 mL) and the mixture heated to 55° C. After 24 hours the mixture was cooled to room temperature and washed with water (3×20 mL), dried (Na₂SO₄) and evaporated under reduced pressure. Trituration with petrol-diethyl ether followed by filtration gave compound 50 (5.05 g).

### Compound 51

### 1,2-O-(exo-1-Methoxyethylidene)-3,4,6-tri-O-benzyl-β-D-mannopyranose

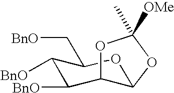

Sodium methoxide (0.452 g) was added to a stirred mixture of compound 50 (30 g) in methanol (250 mL). After 3 hours the mixture was evaporated under reduced pressure and N,N-dimethylformamide (200 mL) added. The mixture was cooled to 0° C. and sodium hydride (15.0 g of a 60% suspension in oil) was added in three lots at 15 minute intervals. After 1 hour benzyl bromide (40 mL) was added dropwise. After 24 hours methanol was added dropwise until the mixture became clear. Ethyl acetate (500 mL) was added and the mixture was washed with water (5×100 mL) and brine, dried (Na₂SO₄) and evaporated under reduced pressure. Trituration with petrol-diethyl ether followed by filtration gave compound 51 (34.6 g).

### Compound 52

### 1,2-Di-O-acetyl-3,4,6-tri-O-benzyl-α-D-mannopyranose

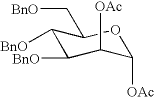

Water (300 mL) was added to a stirred mixture of compound 51 (23 g) and acetic acid (450 mL). After 6 hours ethyl acetate and water were added, the organic phase was separated and the aqueous phase was extracted with ethyl acetate. The combined organic fractions were washed with water, saturated sodium hydrogen carbonate, water and brine, dried (MgSO₄) and evaporated under reduced pressure. Pyridine (150 mL) was added followed by acetic anhydride (17.8 g) and the mixture stirred under argon overnight. Ethyl acetate (300 mL) was added and the mixture washed with 20% hydrochloric acid, saturated sodium hydrogen carbonate and water, dried (MgSO₄) and evaporated under reduced pressure to give compound 52 (22.7 g) which was used without further purification.

### Compound 53

### 2-O-Acetyl-3,4,6-tri-O-benzyl-D-mannopyranose

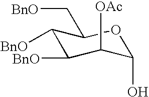

Benzylamine (3.0 g) was added to a solution of compound 52 (10 g) in tetrahydrofuran (100 mL) and the mixture stirred overnight. 2M Hydrochloric acid (50 mL) was added followed by ethyl acetate. The organic phase was separated and the aqueous phase extracted with ethyl acetate. The combined organic fractions were washed with 1M hydrochloric acid and brine, dried (MgSO₄) and evaporated under reduced pressure. Chromatography [SiO₂, ethyl acetate:petrol (20:80)] gave compound 53 (8.9 g).

### Compound 54

### 2-O-Acetyl-3,4,6-tri-O-benzyl-α-D-mannopyranosyl trichloroacetimidate

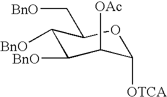

Compound 53 (4.2 g), trichloroacetonitrile (8.5 mL) and activated powdered molecular sieves (˜1 g) were stirred in dichloromethane (40 mL) under argon. After 1 hour 1,8-diazabicyclo[5.4.0]undec-7-ene (0.25 mL) was added. After 1.5 hours the mixture was filtered (Celite), evaporated under reduced pressure and chromatographed [SiO₂, ethyl acetate:petrol:triethylamine (33:66:1)] to give compound 54 (5.32 g).

### Compound 55

### Ethyl 2,3,4,6-tetra-O-acetyl-1-thio-β-D-glucopyranoside

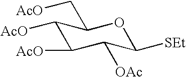

Ethane thiol (2.46 mL) was added to a stirred solution of glucose pentaacetate (10 g) in dry dichloromethane (40 mL). The mixture was cooled to 0° C. and boron trifluoride etherate (3.9 mL) was added dropwise and the mixture stirred overnight. Saturated sodium hydrogen carbonate was added and the organic phase separated. The aqueous phase was extracted with dichloromethane (2×20 mL). The combined organic-fractions were washed with water and brine, dried (MgSO₄) and evaporated under reduced pressure. Trituration with petrol-diethyl ether gave compound 55 (7.2 g).

### Compound 56

### Ethyl 1-thio-β-D-glucopyranoside

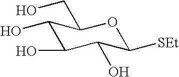

Sodium methoxide was added to a stirred mixture of compound 55 (20 g) in dry methanol (125 mL). After 4 hours Dowex resin (50×2) was added to neutralise the mixture. The mixture filtered and the filtrate evaporated under reduced pressure to give compound 56 (11.1 g).

### Compound 57

### Ethyl 4,6-O-benzylidene-1-thio-β-D-glucopyranoside

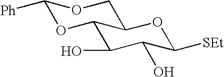

Benzaldehyde dimethyl acetal (14.9 g) and camphorsulfonic acid (1.13 g) were added to a mixture of compound 56 (11 g) and dry N,N-dimethylformamide (50 mL). The mixture was heated to 60° C. at 189 mbar for 4 hours. Excess triethylamine was added and the mixture evaporated under reduced pressure. Ethyl acetate was added and the mixture washed with saturated sodium hydrogen carbonate, water and brine, dried (MgSO₄) and evaporated under reduced pressure. Crystallisation from ethyl acetate-petrol (2 crops) gave compound 57 (10.5 g).

### Compound 58

### Ethyl 2-O-acetyl-3,4,6-tri-O-benzyl-α-D-mannopyranosyl-(1→3)-4,6-O-benzylidene-1-thio-β-D-glucopyranoside

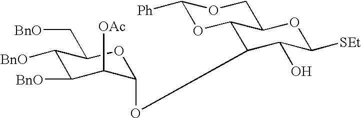

Compound 54 (5.48 g), compound 57 (2.44 g) and activated powdered molecular sieves (˜1.5 g) were stirred in dichloromethane (300 mL) for 1 hour. The mixture was cooled to −78° C. and trimethylsilyl triflate (0.141 mL) added. After 18 hours the mixture was allowed to warm to room temperature and filtered (Celite). The filtrate was washed with saturated sodium hydrogen carbonate and brine, dried (MgSO₄) and evaporated under reduced pressure. Crystallisation from ethyl acetate-petrol gave compound 58 (1.99 g). The mother liquor was chromatographed [SiO₂, ethyl acetate:petrol (10:90) to (30:70)] the product and crystallised from ethyl acetate-petrol to give more compound 58 (0.42 g).

### Compound 59

### Ethyl 2-O-acetyl-3,4,6-tri-O-benzyl-α-D-mannopyranosyl-(1→3)-4,6-O-benzylidene-2-O-levulinyl-1-thio-β-D-glucopyranoside

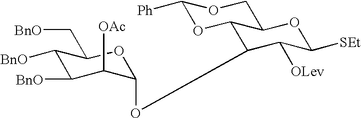

Compound 58 (3.25 g), N,N-dicyclohexylcarbodiimide (1.7 g), 4-dimethylaminopyridine (0.05 g) and levulinic acid (0.959 g) were stirred in dichloromethane under argon. After 20 hours 4-dimethylaminopyridine (0.05 g) was added and after a further 6 hours levulinic anhydride (prepared from levulinic acid (1.92 g) and N,N-dicyclohexylcarbodiimide (1.7 g) in dichloromethane (15 mL)) and triethylamine (5 mL) were added. After 3 days the mixture was filtered (Celite) and washed with water. The aqueous fraction was extracted with dichloromethane and the combined organic fractions were washed with saturated sodium hydrogen carbonate and brine, dried (MgSO₄) and evaporated under reduced pressure. Chromatography [SiO₂, ethyl acetate:petrol (30:70)] gave compound 59 (3.65 g).

### Compound 60

### 1,3,4,6-Tetra-O-acetyl-2-deoxy-2-phthalimido-D-glucopyranose

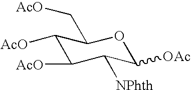

Sodium methoxide (13.5 g) was added to a stirred suspension of D-(+)-glucosamine hydrochloride (53.9 g) in methanol. After 1 hour the reaction was placed in a cold water bath and phthalic anhydride (36.9 g) added. Triethylamine (34.7 mL) was then added over 15 minutes. After 2 days the precipitate was collected by filtration and washed with methanol (2×20 ml) and evaporated under reduced pressure. Pyridine (675 mL) was added and the stirred mixture cooled to 0° C. and acetic anhydride (675 mL) added over 1 hour. After 1 day at room temperature the mixture was cooled to 0° C. and ethanol (270 mL) added over 30 minutes. After 3 hours the mixture was evaporated under reduced pressure and dissolved in dichloromethane (1 L). The mixture was washed with 1M hydrochloric acid (2×500 mL) and saturated sodium hydrogen carbonate (2×500 mL), dried (MgSO₄) and evaporated under reduced pressure. Recrystallisation from methanol gave compound 60 (41.8 g). The mother liquors were evaporated under reduced pressure and chromatographed [SiO₂, ethyl acetate:petrol (50:50) to give further compound 60 (35.1 g).

### Compound 61

### para-Methoxyphenyl 3,4,6-tri-O-acetyl-2-deoxy-2-phthalimido-β-D-glucopyranoside

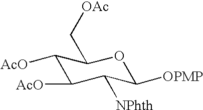

Boron trifluoride etherate (40.4 mL) was added to a stirred solution of compound 60 (76.9 g) and para-methoxyphenol (50 g) in dichloromethane (950 mL) at 0° C. After 1 hour the mixture was allowed to warm to room temperature. After 24 hours the mixture was washed successively with water (×2), 1M sodium hydroxide (×2), water (×2) and brine, dried (MgSO₄) and evaporated under reduced pressure. Crystallisation from ethyl acetate-petrol gave compound 61 (62.5 g).

### Compound 62

### Para-Methoxyphenyl 4,6-O-benzylidene-2-deoxy-2-phthalimido-β-D-glucopyranoside

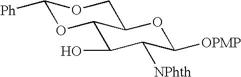

Sodium methoxide (0.5 g) was added to a stirred solution of compound 61 (33.2 g) in methanol (450 mL). After 20 hours Dowex 50WX2 (˜2 spatulas) was added. After 30 minutes the solution was neutral. Methanol (100 mL) was added and the mixture warmed to dissolve the precipitate, filtered and evaporated under reduced pressure. Toluene (50 mL) was added and the mixture evaporated under reduced pressure. Acetonitrile (350 mL), benzaldehyde dimethyl acetal (20.2 mL) and para-toluenesulfonic acid (2.41 g) were added and the mixture stirred. After 20 hours triethylamine (4.7 mL) was added and the mixture evaporated under reduced pressure and crystallised from methanol to give compound 62 (25.5 g).

### Compound 63

### Para-Methoxyphenyl 3-O-benzyl-4,6-O-benzylidene-2-deoxy-2-phthalimido-β-D-glucopyranoside

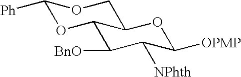

Sodium hydride (1.78 g of a 60% suspension in oil) was added to a stirred solution of compound 62 (15 g) in N,N-dimethylformamide (80 mL) at 0° C. under argon. After 15 minutes benzyl bromide (7.1 mL) was added and the mixture allowed to warm to room temperature. After 6 hours sodium hydride (1.0 g of a 60% suspension in oil) and benzyl bromide (4.0 mL) were added. After 24 hours methanol (10 mL) was added and the mixture evaporated under reduced pressure. The product was dissolved in ethyl acetate and washed with brine (×3), dried (MgSO₄) and evaporated under reduced pressure. Chromatography [SiO₂, ethyl acetate:petrol (30:70) to (40:60)] gave a solid. This was recrystallised from ethyl acetate-petrol to give compound 63 (12 g). A second crop of compound 63 was taken (1.8 g).

### Compound 64

### para-Methoxyphenyl 3,6-di-O-benzyl-2-deoxy-2-phthalimido-β-D-glucopyranoside

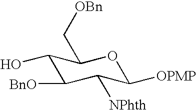

Compound 63 (22.7 g) was added to a stirred mixture of activated powdered molecular sieves (˜5 g) in tetrahydrofuran (800 mL) at 0° C. under argon. After 1 hour sodium cyanoborohydride (50 g) and methyl orange (1 speck) were added. 4M Hydrogen chloride in 1,4-dioxane was added as rapidly as the effervescence allowed until a permanent pink colour developed (1170 mL). After 24 hours the mixture was poured into ice-water and then filtered (Celite). The mixture was extracted with dichloromethane (×2) and the combined extracts stirred with 2M hydrochloric acid (˜200 mL) for 24 hours. The organic phase was then separated and washed with saturated sodium hydrogen carbonate (×2) and water, dried (MgSO₄) and evaporated under reduced pressure. Chromatography [SiO₂, ethyl acetate:petrol (30:70)] gave compound 64 (21.2 g).

### Compound 65

### para-Methoxyphenyl 3,6-di-O-benzyl-4-O-levulinyl-2-deoxy-2-phthalimido-β-D-glucopyranoside

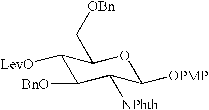

N,N-Dicyclohexylcarbodiimide (11.8 g) was added to a solution of levulinic acid (13.3 g) in dichloromethane (80 mL) at 0° C. After 10 minutes the mixture was allowed to warm to room temperature and stirred for 3 hours. The mixture was then filtered into a solution of compound 64 (6.8 g) in pyridine (70 mL), washing through with dichloromethane (20 mL). After 3 days the mixture was poured into ice-water and stirred for 30 minutes. The mixture was extracted with dichloromethane and the extracts washed with 2M hydrochloric acid (×2) saturated sodium hydrogen carbonate (×2) and water, dried (MgSO₄) and evaporated under reduced pressure. Chromatography [SiO₂, ethyl acetate:petrol (40:60) to (50:50)] gave compound 65 (7.83 g).

### Compound 66

### 3,6-Di-O-benzyl-4-O-levulinyl-2-deoxy-2-phthalimido-D-glucopyranose

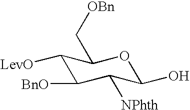

Ceric ammonium nitrate (28.1 g) was added to a vigorously stirred mixture of compound 65 (7.82 g) in toluene (115 mL), acetonitrile (84 mL) and water (37 mL). After 2 hours ethyl acetate was added and the mixture washed with water (×2) and the combined aqueous extracts re-extracted with ethyl acetate. The combined organic fractions were washed with saturated sodium hydrogen carbonate and brine, dried (MgSO₄) and evaporated under reduced pressure to give compound 66 (6.64 g).

### Compound 67

### 3,6-Di-O-benzyl-4-O-levulinyl-2-deoxy-2-phthalimido-β-D-glucopyranosyl trichloroacetimidate

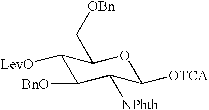

Trichloroacetonitrile (11.3 mL) was added to a stirred mixture of compound 66 (6.64 g) and activated powdered molecular sieves (˜2 g) in dichloromethane (71 mL). After 2 hours 1,8-diazabicyclo[5.4.0]undec-7-ene (0.56 mL) was added. After 1 hour the mixture was filtered (Celite) and evaporated under reduced pressure. Chromatography [SiO₂, ethyl acetate:petrol:triethylamine (44:55:1)] gave compound 67 (5.63 g).

### Compound 68

### para-Methoxyphenyl 3,6-di-O-benzyl-4-O-levulinyl-2-deoxy-2-phthalimido-β-D-glucopyranosyl-(1→4)-3,6-di-O-benzyl-2-deoxy-2-phthalimido-β-D-glucopyranoside

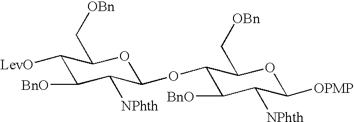

Compound 67 (7.94 g), compound 64 (6.45 g) and activated powdered molecular sieves (˜3 g) were stirred in dichloromethane (155 mL) for 30 minutes. The mixture was then cooled to −78° C. and trimethylsilyl triflate (0.196 mL) was added. After 4 hours the mixture was allowed to warm to room temperature and filtered (Celite). The filtrate was washed with saturated sodium hydrogen carbonate and brine, dried (MgSO₄), evaporated under reduced pressure and chromatographed [SiO₂, (30:70) to (40:60)] to give compound 68 (2.72 g).

### Compound 69

### para-Methoxyphenyl 3,6-di-O-benzyl-2-deoxy-2-phthalimido-β-D-glucopyranosyl-(1→4)-3,6-di-O-benzyl-2-deoxy-2-phthalimido-β-D-glucopyranoside

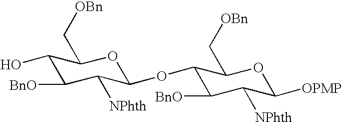

Acetic acid (46 mL) and hydrazine monohydrate (3.2 mL) were added to a stirred mixture of compound 68 (7.32 g) in pyridine (178 mL). After 50 minutes ethyl acetate was added and the mixture washed with saturated sodium hydrogen carbonate (×3) and brine. The aqueous extracts were extracted with ethyl acetate and the combined organic fractions were washed with saturated sodium hydrogen carbonate and brine, dried (MgSO₄) and evaporated under reduced pressure. Toluene was added and the mixture was evaporated under reduced pressure. The addition of toluene and evaporation under reduced pressure was repeated twice. Dichloromethane was added and the mixture was evaporated under reduced pressure. The addition of dichloromethane and evaporation under reduced pressure was repeated twice. Chromatography [SiO₂, ethyl acetate:petrol (30:70) to (50:50)] gave compound 69 (6.36 g).

### Compound 70

### para-Methoxyphenyl 2-O-acetyl-3,4,6-tri-O-benzyl-α-D-mannopyranosyl-(1→3)-4,6-O-benzylidene-2-O-levulinyl-β-D-glucopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside

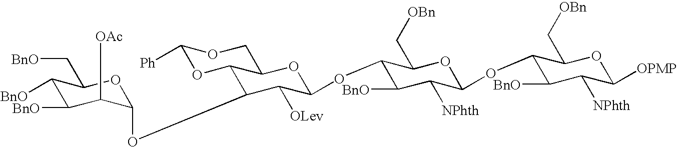

Compound 59 (3.65 g), compound 69 (4.36 g) and powdered activated molecular sieves (˜3 g) were stirred in dichloromethane (500 mL) under argon. After 1 hour methyl triflate (2.17 mL) was added. After 22 hours triethylamine (30 mL) was added. After 15 minutes the mixture was filtered (Celite) and the filtrate washed with water and brine, dried (MgSO₄) and evaporated under reduced pressure. Chromatography [SiO₂, ethyl acetate:petrol (30:70) to (40:60)] gave impure compound 70. Dichloromethane (35 mL), 4-dimethylaminopyridine (0.43 g), triethylamine (5 mL) and 4-hexyl-benzoyl chloride (1.56 mL) were added. After 20 hours ethyl acetate, water and brine were added. The organic fraction was separated and the aqueous fraction was extracted with ethyl acetate. The combined organic fractions were washed with 1M hydrochloric acid, saturated sodium hydrogen carbonate and brine, dried (MgSO₄) and evaporated under reduced pressure. Chromatography [SiO₂, ethyl acetate:petrol (30:70) to (50:50)] gave compound 70 (5.09 g).

### Compound 71

### para-MethoxyPhenyl 2-O-acetyl-3,4,6-tri-O-benzyl-α-D-mannopyranosyl-(1→3)-4,6-O-benzylidene-β-D-glucopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside

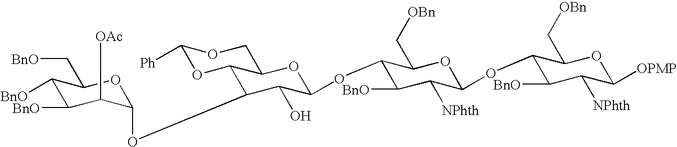

Hydrazine acetate (0.86 g) was added to a mixture of compound 70 (5.9 g) and methanol (250 mL) and the mixture heated at 60° C. overnight. The mixture was cooled and saturated sodium hydrogen carbonate (100 mL) and dichloromethane (200 mL) added. The organic phase was separated and the aqueous phase extracted with dichloromethane (2×100 mL). The combined extracts were washed with brine (100 mL), dried (MgSO₄) and evaporated under reduced pressure. Chromatography [SiO₂, ethyl acetate:petrol (50:50)] gave compound 71 (4.0 g).

### Compound 72

### para-Methoxyphenyl 2-O-acetyl-3,4,6-tri-O-benzyl-α-D-mannopyranosyl-(1→3)-4,6-O-benzylidene-2-O-acetyl-β-D-mannopyranosyl-(1→4)-3,6-di-O-benzyl-2-deoxy-2-phthalimido-β-D-glucopyranosyl-(1-4)-3,6-di-O-benzyl-2-deoxy-2-phthalimido-β-D-glucopyranoside

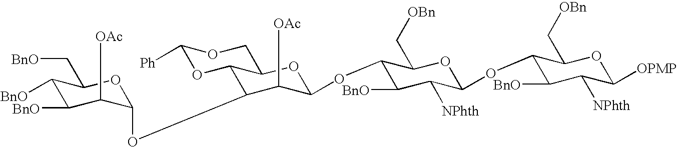

Compound 71 (4.4 g) and anhydrous pyridine (6.3 mL) were dissolved in dichloromethane (50 mL). The mixture was cooled to 0° C. and triflic anhydride (5.6 mL) was added. The mixture was allowed to warm to room temperature over 2 hours and dichloromethane and saturated sodium hydrogen carbonate were added. The organic phase was separated, dried (MgSO₄) and evaporated under reduced pressure. Toluene (100 mL) and tetra-butylammonium acetate (5.1 g) were added and the reaction vessel placed in a sonic bath for 16 hours. The mixture was evaporated under reduced pressure and chromatographed [SiO₂, ethyl acetate:petrol (50:50)] to give compound 72 (3.0 g).

### Compound 73

### Para-Methoxyphenyl 2-O-acetyl-3,4,6-tri-benzyl-α-D-mannopyranosyl-(1→3)-2-O-acetyl-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside

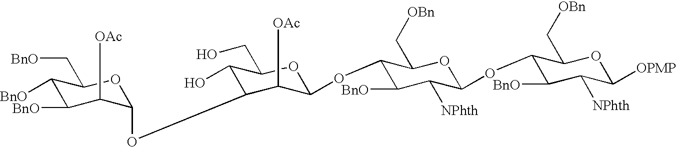

para-Toluenesulfonic acid (0.032 g) was added to a mixture of compound 72 (3.09 g) in methanol (50 mL) and 1,4-dioxane (30 mL). The mixture was heated at 85° C. for 2 hours and then cooled to room temperature. Water (100 mL) and dichloromethane (100 mL) were added and the organic phase separated. The aqueous phase was extracted with dichloromethane (2×50 mL) and the combined organic fractions were washed with sodium hydrogen carbonate (100 mL) and brine (100 mL), dried (MgSO₄) and evaporated under reduced pressure. Chromatography [SiO₂, ethyl acetate:petrol (50:50) to (67:33)] gave compound 73 (1.5 g).

### Compound 74

### para-Methoxyphenyl 2-O-acetyl-3,4,6-tri-O-benzyl-α-D-mannopyranosyl-(1→6)-[2-O-acetyl-3,4,6-tri-O-benzyl-α-D-mannopyranosyl-(1→3)]-2-O-acetyl-β-D-mannopyranosyl-(1→4)-3,6-di-O-benzyl-2-deoxy-2-phthalimido-β-D-glucopyranosyl-(1→4)-3,6-di-O-benzyl-2-deoxy-2-phthalimido-β-D-glucopyranoside

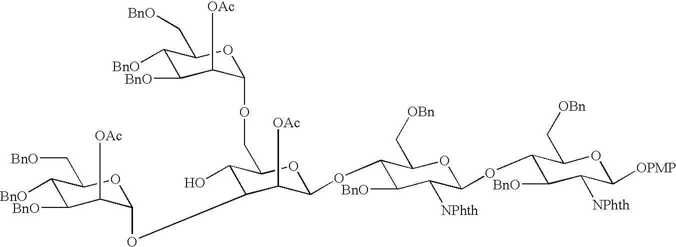

Compound 73 (0.500 g), compound 6 (0.184 g) and activated powdered molecular sieves were stirred in dry dichloromethane (30 mL) under argon. The mixture was cooled to −40° C. and trimethylsilyl triflate (0.003 mL) was added. After 2 hours the mixture was filtered (Celite) and the filtrate washed with saturated sodium hydrogen carbonate and brine, dried (MgSO₄) and evaporated under reduced pressure. Chromatography [SiO₂, ethyl acetate:petrol (33:67) to (50:50)] gave compound 74 (0.40 g).

### Compound 75

### 2-O-Acetyl-3,4,6-tri-O-benzyl-α-D-mannopyranosyl-(1→6)-[2-O-acetyl-3,4,6-tri-O-benzyl-α-D-mannopyranosyl-(1→3)]-2,4-di-O-acetyl-β-D-mannopyranosyl-(1→4)-3,6-di-O-benzyl-2-deoxy-2-phthalimido-β-D-glucopyranosyl-(1→4)-1-O-acetyl-3,6-di-O-benzyl-2-deoxy-2-phthalimido-β-D-glucopyranoside

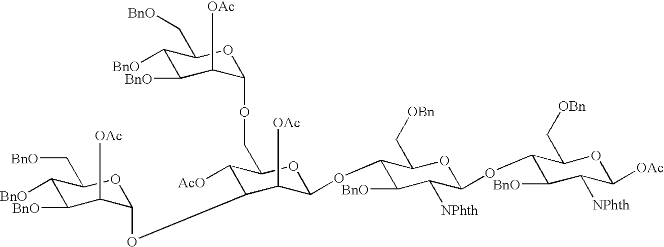

Ceric ammonium nitrate (0.185 g) was added to a vigorously stirred mixture of compound 74 (0.15 g) in acetonitrile (3 mL), toluene (2 mL) and water (1 mL). After 25 minutes ethyl acetate was added and the mixture washed with water, saturated sodium hydrogen carbonate and brine, dried (MgSO₄) and evaporated under reduced pressure. Dichloromethane (2 mL) and pyridine (2.2 mL) were added and acetic anhydride (0.7 mL) was then added dropwise. The mixture was stirred for 20 hours and dichloromethane added. The mixture was washed with saturated sodium bicarbonate, water and brine, dried (MgSO₄) and evaporated under reduced pressure. Chromatography [SiO₂, ethyl acetate:petrol (50:50)] gave compound 75 (0.127 g).

### Compound 76

### Benzyl 2-O-acetyl-3,4,6-tri-O-benzyl-α-D-mannopyranosyl-(1→6)-[2-O-acetyl-3,4,6-tri-O-benzyl-α-D-mannopyranosyl-(1→3)]-2,4-di-O-acetyl-β-D-mannopyranosyl-(11-4)-3,6-di-O-benzyl-2-deoxy-2-phthalimido-β-D-glucopyranosyl-(1→4)-3,6-di-O-benzyl-2-deoxy-2-phthalimido-1-thio-β-D-glucopyranoside

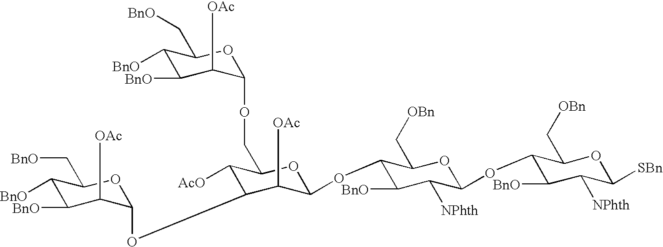

Boron trifluoride etherate (0.009 mL) was added to a stirred mixture of compound 75 (0.123 g) and benzyl mercaptan (0.013 mL) in dry dichloromethane (2 mL). After 2 hours dichloromethane (50 mL) was added and the mixture was washed with saturated sodium hydrogen carbonate, water and brine, dried (MgSO₄) and evaporated under reduced pressure. Radial chromatography [SiO₂, ethyl acetate:petrol (40:60)] gave compound 76 (0.103 g).

### Compound 77

### Benzyl 2-O-acetyl-3,4,6-tri-O-benzyl-α-D-mannopyranosyl-(1→6)-[2-O-acetyl-3,4,6-tri-O-benzyl-α-D-mannopyranosyl-(1→3)]-2,4-di-O-acetyl-β-D-mannopyranosyl-(1→4)-3,6-di-O-benzyl-2-deoxy-2-acetamido-β-D-glucopyranosyl-(1→4)-3,6-di-O-benzyl-2-deoxy-2-acetamido-1-thio-β-D-glucopyranoside

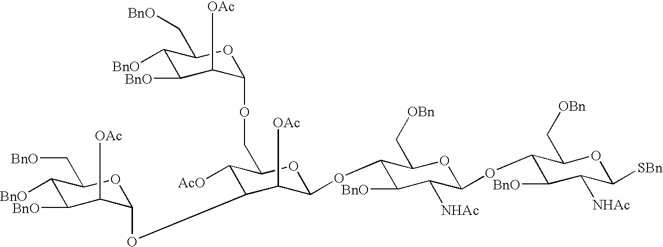

Ethylene diamine (2 mL) was added to a stirred mixture of compound 76 (0.096 g) and n-butanol (10 mL) under argon and the mixture heated to 80° C. After 20 hours the mixture was evaporated under reduced pressure. Toluene (20 mL) was added and the mixture was evaporated under reduced pressure. The addition of toluene and evaporation under reduced pressure was repeated twice. Pyridine (5 mL) and acetic anhydride (1 mL) were added and the mixture stirred under argon for 16 hours. Ethyl acetate was added and the mixture was washed with saturated sodium bicarbonate, water and brine, dried (MgSO₄) and evaporated under reduced pressure. Chromatography [SiO₂, ethyl acetate:petrol (70:30)] gave compound 77 (0.069 g).

### Compound 78

### α-D-Mannopyranosyl-(1→6)-[α-D-mannopyranosyl-(1→3)]-β-D-mannopyranosyl-(1→4)-2-deoxy-2-acetamido-β-D-glucopyranosyl-(1→4)-2-deoxy-2-acetamido-1-thio-β-D-glucopyranose

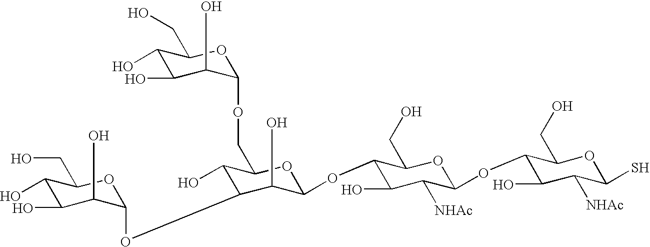

Sodium (0.010 g) was added to liquid ammonia (10 mL) at −78° C. After 10 minutes compound 77 (0.030 g) in tetrahydrofuran (2 mL) was added. After 30 minutes ammonium chloride (0.038 g) was added and the mixture allowed to warm to room temperature. Water (1 mL) was added and the mixture size exclusion chromatographed [Biorad P2 gel, 0.04M ammonium carbonate] and lyophilised to give compound 78 (0.009 g).

### Compound 79

### Disulfide of α-D-Mannopyranosyl-(1→6)-[α-D-mannopyranosyl-(1→3)]-β-D-mannopyranosyl-(1→4)-2-deoxy-2-acetamido-β-D-glucopyranosyl-(1→4)-2-deoxy-2-acetamido-1-thio-β-D-glucopyranose

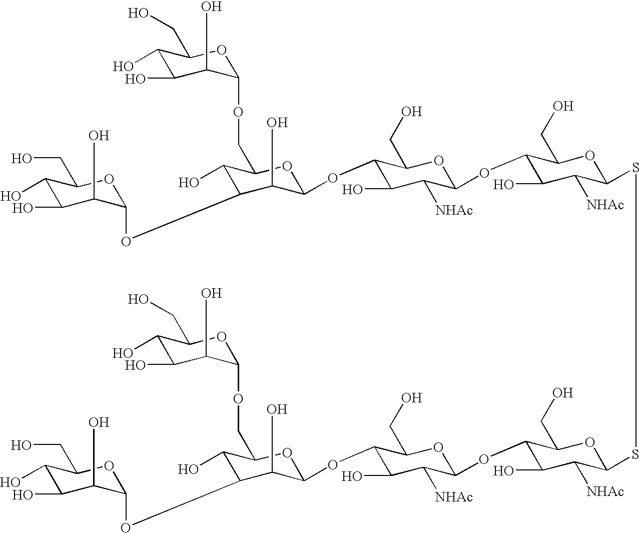

Compound 78 in water was allowed to stand under an air atmosphere until the thiol was completely converted to the disulfide (about 5 days).

Synthesis of SBL-Compound 78 Conjugate (Inactivated with PMSF).

Phenylmethylsulfonyl fluoride (PMSF) (6 μL of a 1.0 M solution in ethanol) was added to a solution of Subtilisin Bacillus lentus (SBL) S156C mutant (0.3 g) in ammonium carbonate buffer (pH 8.6) (100 μL). After 5 minutes the mixture was desalted on a Zeba Desalt Spin Column (Pierce) that had been pre-equilibrated with 50 mM ammonium carbonate buffer (pH 8.6). Compound 31 (25 μL of a 4 mg/mL solution in water) was added. After 2 hours a 5 μL aliquot was taken and analysed by mass spectrometry (ESI-TOF), showing conversion to the SBL-compound 78 conjugate (inactivated with PMSF) (observed mass 27792, theoretical 27793).

1. A process for the preparation of a thiosaccharide represented by
Saccharide-S-H
wherein Saccharide comprises at least 4 sugar units, and wherein said process comprises subjecting a corresponding compound of the formula
(P)Saccharide-S-(P)
wherein (P) represents an O- or S-protecting group(s), to Birch reduction.

2. The process according to claim 1, wherein (P) is acyl or benzyl.

3. The process according to claim 1, wherein (P) is acetyl or benzyl.

4. The process according to claim 1, wherein Saccharide-S-H is α-D-Mannopyranosyl-(1→6)-[α-D-mannopyranosyl-(1→3)]-β-D-mannopyranosyl-(1→4)-2-deoxy-2-acetamido-β-D-glucopyranosyl-(1→4)-2-deoxy-2-acetamido-1-thio-β-D-glucopyranose (Compound 78.

5. The process according to claim 1, wherein Saccharide-S-H is {2-Deoxy-2-acetamido-β-D-glucopyranosyl-(1→4)-[2-deoxy-2-acetamido-β-D-glucopyranosyl-(1→2)]-α-D-mannopyranosyl-(1→3)}-{2-deoxy-2-acetamido-β-D-glucopyranosyl-(1→6)-[2-deoxy-2-acetamido-β-D-glucopyranosyl-(1→2)]-α-D-mannopyranosyl-(1→6)}-β-D-mannopyranosyl-(1→4)-2-deoxy-2-acetamido-β-D-glucopyranosyl-(1→4)-[α-L-fucopyranosyl-(1→6)]-2-deoxy-2-acetamidomido-1-thio-β-D-glucopyranose (Compound 47).

6. The process according to claim 1, wherein the process is used for the preparation of a glycoprotein represented by
Saccharide-S-S-Protein
wherein the process comprises converting Saccharide-SH to Saccharide-S-Se-R, and reacting the latter with Protein-SH, wherein R is an organic group.

7. The process according to claim 1 wherein the process is used for the preparation of a glycoprotein represented by
Saccharide-S-S-Protein
wherein the process comprises reacting the Saccharide-SH with Protein-S-Se-R, wherein R is an organic group.

8. The process according to claim 6, wherein the process is used for the preparation of a glycoprotein represented by
Saccharide-S-S-Protein
wherein the process comprises allowing Saccharide-SH to dimerise, and reacting the dimer Saccharide-S-S-Saccharide with Protein-SH.

9. A compound which is {2-Deoxy-2-acetamido-β-D-glucopyranosyl-(1→4)-[2-deoxy-2-acetamido-β-D-glucopyranosyl-(1→2)]-α-D-mannopyranosyl-(1→3)}-[2-deoxy-2-acetamido-β-D-glucopyranosyl-(1→6)-[2-deoxy-2-acetamido-β-D-glucopyranosyl-(1→2)]-α-D-mannopyranosyl-(1∵6)]-β-D-mannopyranosyl-(1→4)-2-deoxy-2-acetamido-β-D-glucopyranosyl-(1→4)-[α-L-fucopyranosyl-(1→6)]-2-deoxy-2-acetamidomido-1-thio-β-D-glucopyranose (Compound 47).

10. Compound 48 which is the disulfide of {2-Deoxy-2-acetamido-β-D-glucopyranosyl-(1→4)-[2-deoxy-2-acetamido-β-D-glucopyranosyl-(1→2)]-α-D-mannopyranosyl-(1→3)}-{2-deoxy-2-acetamido-β-D-glucopyranosyl-(1→6)-[2-deoxy-2-acetamido-β-D-glucopyranosyl-(1→2)]-α-D-mannopyranosyl-(1→6)}-β-D-mannopyranosyl-(1→4)-2-deoxy-2-acetamido-β-D-glucopyranosyl-(1→4)-[α-L-fucopyranosyl-(1→6)]-2-deoxy-2-acetamidomido-1-thio-β-D-glucopyranose (Compound 47).

11. The process according to claim 1, wherein Saccharide comprises at least 5 sugar units.

12. The process according to claim 3, wherein Saccharide comprises at least 5 sugar units.
